(19) United States
(12) Patent Application Publication
(10) Pub. No.: US 2026/0283483 A1
(43) Pub. Date: Sep. 24, 2026

(54) Wireless Patient Monitoring

(71) Applicant: CAMBRIDGE ENTERPRISE LIMITED, Cambridge, Cambridgeshire (GB)

(72) Inventors: Oliver Bonner, Cambridge, Cambridgeshire (GB); Kathryn Beardsall, Cambridge, Cambridgeshire (GB); Christopher Ferris, Cambridge, Cambridgeshire (GB); Peter Money, Cambridge, Cambridgeshire (GB); Gabrielle Whitworth-Smith, Cambridge, Cambridgeshire (GB)

(21) Appl. No.: 19/476,511

(22) PCT Filed: Apr. 25, 2024
(86) PCT No.: PCT/GB2024/051075

§ 371 Date: Oct. 17, 2025

(30) Foreign Application Priority Data
Apr. 28, 2023 (GB) 2306334.0

(51) Int. Cl. A61B 5/0205; A61B 5/00; A61B 5/1455; A61B 5/327; A61B 5/339

(52) U.S. Cl. CPC A61B 5/02055; A61B 5/0006; A61B 5/0008; A61B 5/14551; A61B 5/327; A61B 5/339; A61B 2562/0271

(57) ABSTRACT

Devices and methods for wireless patient monitoring are described. A receiver connectable to a patient monitor comprises a processor and circuitry coupled to the processor. The circuitry is configured to wirelessly receive data based on an output from a pulse oximeter unit, wherein the data comprises a first data set comprising voltage values corresponding to a photodiode current sensed responsive to a first LED of the pulse oximeter unit, and a second data set comprising voltage values corresponding to a photodiode current sensed responsive to a second LED of the pulse oximeter unit. The circuitry is further configured to generate a signal based on the received data, wherein the signal is configured to emulate a wired connection input signal to the patient monitor, and output the generated signal to the patient monitor.

### TECHNICAL FIELD

The present disclosure relates to devices and methods for wirelessly connecting at least one patient vital sign sensing unit to a patient monitor.

### BACKGROUND

Sensors for monitoring patient vital signs, such as sensors for monitoring a patient's blood oxygen saturation (SpO₂), heart rhythm and electrical activity, respiratory rate and skin temperature, etc., are conventionally attached to the patient and are connected via a wire to a patient monitor that displays information representative of the vital signs measured by the sensors. In order for the vital signs of the patient to be accurately monitored, the patient monitor continuously receives the data measured by the sensors. However, the wires connecting the sensors to the patient monitor may be easily dislodged by movement of the patient or by interactions of a medical professional or care giver with the patient, which may interrupt the continuous receiving of vital sign information by the patient monitor.

### SUMMARY OF INVENTION

Embodiments of the invention are set out in the appended claims.

### BRIEF DESCRIPTION OF THE DRAWINGS

Various features of the present disclosure will be apparent from the detailed description which follows, taken in conjunction with the accompanying drawings, which together illustrate features of the present disclosure, and wherein:

FIG. 1 is a schematic diagram of functional blocks of an example receiver.

FIG. 2 is an illustrative diagram of a pulse oximeter unit.

FIG. 3 is an illustrative diagram of a photodiode output response.

FIG. 4 is a schematic diagram of functional blocks of an example receiver configured to emulate a wired connection input signal from a pulse oximeter unit.

FIG. 5 is a schematic diagram of functional blocks of circuitry of an example receiver according to the disclosure.

FIG. 6 is a schematic diagram of functional blocks of example signal generation circuitry of an example receiver according to the disclosure.

FIG. 7 is a schematic diagram of an example circuit of signal generation circuitry of a receiver configured to emulate a signal based on pulse oximeter data.

FIG. 8 is a schematic diagram of an example circuit of signal generation circuitry of a receiver configured to emulate a signal based on pulse oximeter data and to emulate a load on a patient monitor.

FIG. 9 is a schematic diagram of an example circuit of signal generation circuitry of a receiver configured to emulate a signal based on pulse oximeter data and monitor respective first and second LED drive current signals.

FIG. 10 is a schematic diagram of an example circuit of signal generation circuitry of a receiver configured to emulate a signal based on pulse oximeter data and to monitor a feedback signal.

FIG. 11 is a flow chart illustrating an example method performed by a receiver configured to emulate a wired connection input signal from a pulse oximeter unit.

FIG. 12 is a schematic diagram of functional blocks of an example transmitter configured to transmit pulse oximeter data.

FIG. 13 is a flow chart illustrating an example method performed by a transmitter configured to transmit pulse oximeter data.

FIG. 14 is a schematic diagram of functional blocks of an example system.

FIG. 15 is a schematic diagram of functional blocks of another example receiver configured to emulate wired connection input signals from an ECG unit and a temperature sensor unit.

FIG. 16 is a schematic diagram of functional blocks of circuitry of an example receiver configured to emulate wired connection input signals from an ECG unit and a temperature sensor unit.

FIG. 17 is a schematic diagram of functional blocks of example signal generation circuitry of an example receiver according to the disclosure.

FIG. 18 is a schematic diagram of an example circuit of signal generation circuitry of a receiver configured to emulate a signal based on temperature sensor data.

FIG. 19 is a schematic diagram of an example circuit of signal generation circuitry of a receiver configured to emulate a signal based on ECG data.

FIG. 20 is a schematic diagram of an example circuit of signal generation circuitry of a receiver configured to emulate a signal based on ECG data and a lead-off event signal.

FIG. 21 is a flow chart illustrating an example method performed by an example receiver configured to emulate wired connection input signals from an ECG unit and a temperature sensor unit.

FIG. 22 is a schematic diagram of functional blocks of an example transmitter configured to transmit ECG and temperature sensor data.

FIG. 23 is a flow chart illustrating an example method performed by a transmitter configured to transmit ECG and temperature sensor data.

FIG. 24 is a schematic diagram of functional blocks of an example system.

FIG. 25 is a schematic diagram of functional blocks of example signal generation circuitry of an example receiver configured to emulate an impedance signal waveform corresponding to a respiratory measurement.

FIG. 26 is a schematic diagram of an example circuit of signal generation circuitry of a receiver configured to emulate an impedance signal waveform based on a received at least one parameter value.

FIG. 27 is a schematic diagram of an example circuit of signal generation circuitry comprising a resistor network.

FIG. 28 is a flow chart illustrating an example method performed by an example receiver configured to emulate an impedance signal waveform corresponding to a respiratory measurement.

FIG. 29 is a schematic diagram of functional blocks of an example system.

It is to be understood that while the patient monitors shown in the Figures are illustrated as displaying a graphical representation of a vital sign and numerical values of (a) vital sign(s), the patient monitors described herein are not so limited and may be any patient monitor known in the art and may be configured to indicate a vital sign, or any combination of vital signs, in any manner.

### DETAILED DESCRIPTION

During patient treatment, diagnosis or care, a patient's vital signs are often continuously monitored in order to alert a medical professional or care giver to changes in the patient's condition. In order for vital signs to be measured, sensors are attached to a patient, for example, by way of pads using adhesives, or with clips to maintain coupling between a sensor and a contact point, for example on a patient's body. In order to provide information about the monitored vital sign(s) of the patient to a medical professional or care giver, the data collected by the sensor(s) is provided to a patient monitor, which outputs (e.g., displays) an indication that is representative of the vital sign(s).

In some settings, it may be useful to monitor more than one vital sign of a patient at the same time. To enable this, many patient monitors are configured to receive inputs from different vital sign sensors and are configured to simultaneously provide indications of the multiple monitored vital signs of the patient.

However, conventional patient monitors are configured to receive input from the vital sign sensors attached to the patient via wired connections, for example, where the sensor attached to the patient communicates (i.e., sends and receives) data with the patient monitor over a lead that physically extends between an input line (or connector thereof) of the patient monitor and the sensor attached to the patient. The use of a wire, or lead, physically extending between the patient and the patient monitor may restrict the movement of the patient or inhibit interactions between the patient and the medical professional or care giver. The use of such a wire, or lead, may also lead to interruption in the otherwise continuous monitoring of the patient's vital signs if unavoidable, or accidental movement of the patient relative to the patient monitor or intervening bodies (or vice versa) causes one or more of the wires to be dislodged and therefore disconnected from at least one of the sensors attached to the patient and connected to the patient monitor. This issue can be exacerbated when multiple vital sign sensors are attached to the patient and connected to the patient monitor to enable the simultaneous monitoring of multiple vital signs.

The present disclosure provides example receivers connectable to a patient monitor, such as a conventional patient monitor that is configured to receive input from one or more vital sign sensors via a wired connection). The example receivers disclosed herein provide a wireless communication link between the sensors attached to the patient and the patient monitor. The wireless communication link is more robust to patient movement and may prevent signal interruption caused by the dislodging of the connecting wires and/or enable freer movement of the patient and/or interaction between the patient and a medical professional or caregiver. The example receivers disclosed herein are configured to wirelessly receive data from vital sign sensing units (comprising vital sign sensors) attached to the patient, such as a pulse oximeter unit, an electro-cardiogram (ECG or EKG) unit, or a temperature sensor unit.

The receivers disclosed herein are further configured to generate at least one signal based on the wirelessly received data to emulate a wired connection input signal to the patient monitor and output the generated signal(s) to the patient monitor. In this way, the wired connection physically extending between the patient and the patient monitor can be removed without affecting the operation of the patient monitor, such that the receivers disclosed herein may be retrofitted to conventional patient monitors.

The example receivers disclosed herein remove the wired connection between the patient and the patient monitor required in a conventional patient monitoring system, while maintaining the continuous monitoring of the patient's vital signs. This is particularly advantageous in settings where the patient may be prone to unavoidable, or involuntary movements, and where movement of the patient may be required for treatment to be administered or diagnosis to be performed. In particular, it has been found that during the treatment of infants, for example in neonatal care, improved and sustained physical contact, such as skin-to-skin contact, between the infant and another (e.g., human) body, such as that of a relation or caregiver, can significantly increase the improvement in health of the infant. The example receivers disclosed herein may also be particularly advantageous in outpatient settings, or during patient transport, such as in an ambulance where movement may be unavoidable.

It is to be understood that the example receivers disclosed herein may be used with respect to any patient monitoring, including in the treatment or diagnosis of human or animal bodies, and may be used in any clinical, medical or care-giving environment.

In the following description, for the purposes of explanation, numerous specific details of certain examples are set forth. Reference in the specification to “an example” or similar language means that a particular feature, structure, or characteristic described in connection with the example is included in at least that one example, but not necessarily in other examples. It should also be understood that examples may be combined in any combination where feasible.

The following description relates to: receivers configured to wirelessly receive data from a vital sign sensing unit, generate signal(s) based on the wirelessly received data, where the signal(s) is(are) configured to emulate a wired connection input signal(s) to the patient monitor and output the generated signal(s) to the patient monitor; and transmitters configured to wirelessly transmit the data from the vital sign sensing units to the receivers.

It is to be understood that the signals disclosed herein are information carriers and may carry any type of information. For example, the signals disclosed herein may be e.g., voltage, current or impedance signals that carry information pertaining to voltages, currents or impedance, respectively. However, the signals are not so limited and may carry information pertaining, or relating to any variable, parameter, or measurable. It is also to be understood that a signal as disclosed herein may carry information pertaining to multiple different variables, parameters, or measurables. For example, a signal as disclosed herein may carry information relating to two or more measurables etc. It may be that a voltage signal disclosed herein, and e.g., output to the patient monitor, comprises a voltage. For example, a voltage to be applied to an input, e.g., an input line, of the patient monitor. It may be that a current signal disclosed herein, and e.g., output to the patient monitor, comprises a current. For example, a current to be provided to an input, e.g., an input line, of the patient monitor.

Where it is disclosed that the sensor(s) of the sensing unit(s) are attached to the patient, it is to be understood that the sensor(s) are coupled to the patient to provide sensing contact. For example, the sensors are coupled to the patient, or a portion thereof, to enable the at least one vital sign of the patient to be sensed. For example, the sensor(s) may be in physical contact (and in some cases direct physical contact), or for example in conductive contact, such as electrically conductive contact, or heat conductive contact with the patient, or a portion thereof.

It is also to be understood that the signal(s) generated by the receivers disclosed herein are configured to emulate a wired connection input signal(s) to the patient monitor such that the patient monitor receives, and for example processes the signal(s) output by the receivers disclosed herein in the same way that it would receive (and process) signal(s) received via a wired connection from the vital sign sensor(s) or sensing unit(s). In this way it is to be understood that the emulation of the wired connection input signal(s) by the receivers disclosed herein enables the receivers to be retrofitted to conventional patient monitors, which may be expensive and difficult to replace as they may be, or have functionality, integrated with other systems in a clinical, medical, or care giving environment. The emulation of the wired connection input signal(s) by the receivers disclosed herein is to be understood to reproduce the input signal(s) the conventional patient monitor would receive from (a) sensing unit(s) connected to the patient monitor via a wire, or lead etc. Furthermore, it is to be understood that the emulated signal(s) are configured to produce the same effect on the patient monitor as signal(s) received from the sensing unit(s) connected to the patient monitor via a wired connection.

Examples disclosed herein relate to receivers configured to wirelessly receive data from a vital sign sensing unit, generate signal(s) based on the wirelessly received data, where the signal(s) is(are) configured to emulate a wired connection input signal(s) to the patient monitor and output the generated signal(s) to the patient monitor. Examples disclosed herein also relate to transmitters configured to wirelessly transmit data from vita sign sensing units (attached to patients) to the receivers.

The examples enable the data (or information) indicative of the vital sign(s) of the patient to be provided to the patient monitor wirelessly, which improves the reliability of (e.g., continuous) reception of vital sign information by the patient monitor. For example, the wireless reception of the vital sign information enabled by the receivers (and transmitters) disclosed herein reduces the likelihood of the interruption of signals due to disconnection of wires and allows for freer movement of the patient, or a wider range of patient-care giver etc., interactions. The examples disclosed herein further enable these improvements to be achieved with existing patient monitors (and integrated or inter-connected systems) by enabling the receivers to be retrofitted to existing patient monitors that are configured for receiving wired input.

More particularly, an example receiver disclosed herein is configured to wirelessly receive pulse oximeter data based on an output from a pulse oximeter unit and to generate and output to a patient monitor a signal based on the received data that is configured to emulate a wired connection input signal to the patient monitor. This receiver, or another example receiver disclosed herein is configured to wirelessly receive ECG data sensed by an ECG unit and temperature sensor data sensed by a temperature sensor unit and generate and output respective signals based on the received ECG data and the received temperature sensor data to emulate respective wired connection input signals received by the patient monitor from the ECG unit and the temperature sensor unit.

It may be that the receiver configured to wirelessly receive data based on an output from a pulse oximeter (e.g., pulse oximeter data) and the receiver configured to wirelessly receive ECG data sensed by an ECG unit and temperature sensor data sensed by a temperature sensor unit may be the same receiver, or may be separate receivers. For example, at least the signal generation circuitry of the receiver configured to wirelessly receive pulse oximeter data and at least the signal generation circuitry of the receiver configured to wirelessly receive ECG data sensed by an ECG unit and temperature sensor data sensed by a temperature sensor unit may be integrated into a single receiver, and may for example be coupled to the same processor, or may be powered by the same power supply (e.g., battery).

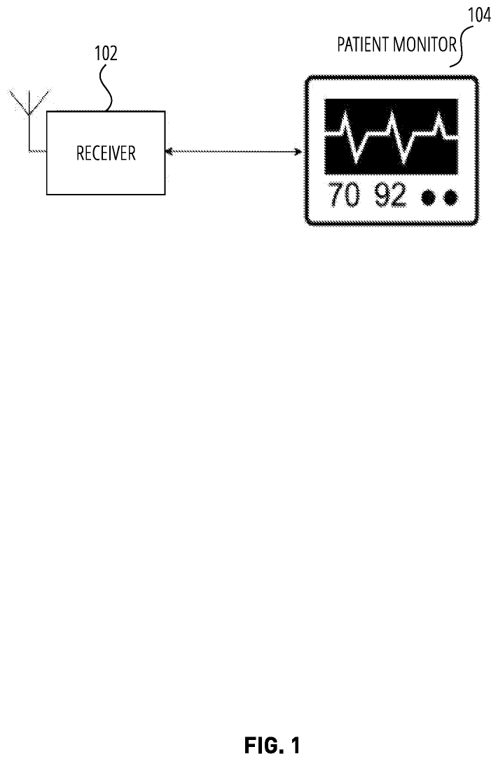

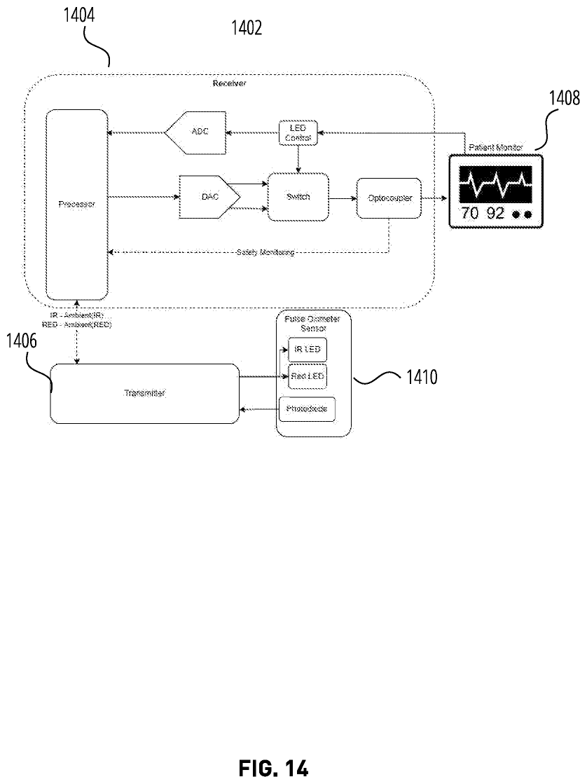

In the following description of the figures, the example receivers of FIG. 1 and FIGS. 4 to 10 and share several features in common. Corresponding features in FIG. 1, and 4 to 10 are referred to using similar reference numerals. The example receivers of FIGS. 14 to 24 also share several features in common and corresponding features in FIGS. 14 to 24 are referred to using similar reference numerals.

FIG. 1 shows a schematic diagram of a receiver 102 connectable to a patient monitor 104.

As described above, the patient monitor may be configured to provide an indication of one or more of a patient's vital signs, e.g., to a medical professional, or care giver. For example, the patient monitor 104 may be configured to display at least one of a graphical representation of a vital sign, such as an electrocardiogram (e.g., an electrogram of the heart), or a plethysmograph (or a photoplethysmograph, i.e., a pulse oximeter output as a function of time, also known as a pleth) etc,. The patient monitor 104 may alternatively, or additionally be configured to indicate, e.g., display, a parameter value indicative of a measurable vital sign, such as a skin temperature reading, a pulse rate, or blood oxygen saturation (SpO₂) value etc,.

The patient monitor 104 is configured to provide the indication of the one or more vital signs based on one or more signals comprising information indicative of the vital sign that is to be received from at least one vital sign sensing unit attached to the patient. The patient monitor 104 is configured to receive said signal(s) via a wired connection from the vital sign sensing unit. The patient monitor 104 comprises one or more input lines, or connectors thereof, (not shown) to receive one or more wires (or leads) to communicatively couple the patient monitor 104 to the one or more vital sign sensing units. For example, to receive the signals comprising the information indicative of the vital sign(s) from the vital sign sensing units.

It may be that the receiver 102 and the patient monitor 104 are connectable by way of one or more wires, or leads, to communicatively couple the receiver 102 and the patient monitor 104. For example, the receiver 102 may be configured to output signals to the patient monitor 104 via one or more wires or leads that are connectable to the connectors of the patient monitor 104. It may be that the receiver 102 is configured to output a signal comprising information corresponding to a given vital sign over one or more wires or leads to a connector of the patient monitor 104 via which the monitor is configured to receive a respective wired connection vital sign input signal. For example, the wiring or lead configuration between the receiver 102 and the patient monitor 104 may reproduce the wiring or lead configuration of a conventional wired patient monitoring system with respect to the vital sign signals and the input connectors of the patient monitor 104. However, in contrast to wired patient monitoring systems, the receiver 102 is not attached to the patient.

It may be that the receiver 102 comprises one or more output connectors (or ports, or output lines not shown) to which wires or leads may be connected in order to connect, and for example to provide communication between, the receiver 102 and the patient monitor 104.

Various example receivers are disclosed below. FIGS. 1 and 4 to 10 show schematic diagrams of example receivers configured to receive data from a pulse oximeter unit. FIGS. 14 to 20 show schematic diagrams of example receivers configured to receive data from ECG and temperature sensing units.

Example receivers configured to wirelessly receive data from a pulse oximeter unit are described below. A brief explanation of the basic operating principles of a pulse oximeter unit is presented in order to aid understanding of the signals received and generated by the example receivers.

A pulse oximeter unit is configured to measure a blood (e.g., an arterial blood) oxygen saturation value in a patient. A pulse oximeter unit comprises a pulse oximeter. It may be that the pulse oximeter is dis-connectable from the pulse oximeter unit. For example, the pulse oximeter may be replaceable or disposable.

During use, a pulse oximeter (e.g., of a pulse oximeter unit), is attached to a portion of a patient's body, for example using a clip or other attaching means, such as a sleeve, or fabric cuff etc. A pulse oximeter (e.g., of a pulse oximeter unit) comprises first and second LEDs, configured to irradiate, or illuminate, a portion of a patient's body. The first and second LEDs are positioned adjacent a first side of the portion of the patient's body. The pulse oximeter further comprises a photodiode positioned adjacent to a second side of the portion of the patient's body, the second side opposing the first side such that, during use, the portion of the patient's body intervenes between the first and second LEDs and the photodiode. The photodiode is configured to detect the radiation emitted from the first and second LEDs after it has passed through the portion of the patient's body. It may be that the first and second LEDs emit radiation at different wavelengths. For example, the first LED may be configured to emit radiation in the infrared wavelength range (for example from approximately 700 nanometers to 1 micrometer, e.g., at approximately 940 nanometers) and the second LED is configured to emit light in the red part of the visible (or optical) wavelength range (for example from approximately 620-700 nanometers, e.g., at approximately 660 nanometers). The photodiode is configured to detect radiation at both wavelengths, or wavelength ranges.

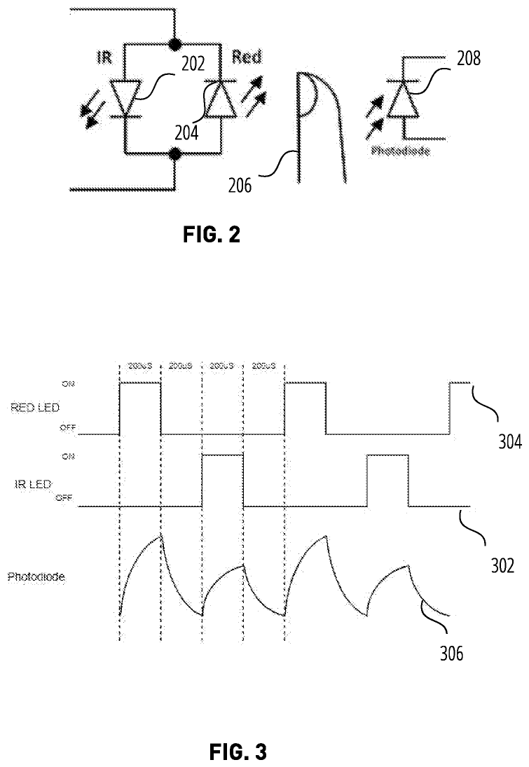

A schematic diagram of an example pulse oximeter is shown in FIG. 2. In the illustrated example, the first LED 202 is configured to emit light in the infrared wavelength range and the second LED is configured to emit light in the red part of the visible (or optical) wavelength range. The first LED 202 and the second LED 204 are positioned on a first side of a patient's digit 206, and the photodiode 208 is positioned on an opposing side of the digit 206 such that the digit 206 is intervening between the respective LEDs and the photodiode and the radiation emitted from the first LED 202 and the second LED 206 that is detected by the photodiode 208 passes through the digit 206 of the patient before being detected.

Although the portion of the patient's body through which the radiation from the respective LEDs 202, 204 travels before detection by the photodiode 208 is illustrated in FIG. 2 as a digit 206, it is not so limited. A pulse oximeter may be attached to, i.e., on opposing sides of, any body part from which an arterial blood flow can be detected (i.e., where there is an artery and where the body part is sufficiently translucent), such as an earlobe, or in the case of infants, a hand or foot.

A blood oxygen saturation value can be calculated using well-known methods based on the relative transmission of the radiation emitted from the first LED 202 and the second LED 204 through the digit 206 (or equivalently, the relative absorption by the digit 206). For example, it is known that oxygenated hemoglobin absorbs more infrared light and is more transmissive to red (optical) light than deoxygenated hemoglobin.

During use, the first LED 202 and the second LED 204 of the pulse oximeter may be pulsed, or otherwise driven, at different intervals separated in time during a cycle, and the response of the photodiode during those intervals may be measured.

An example photodiode response curve is illustrated in FIG. 3, which shows an illustrative first LED 202 drive signal 302 and a second LED 204 drive signal 304, where the respective LEDs are being driven (or powered on and off) at different intervals separated in time. In the response curve illustrated in FIG. 3, the LEDs are driven during 200 microsecond intervals, although this is not intended to be limiting and the time interval may be greater or shorter. A photodiode response curve 306 is also illustrated and can be seen to reflect when the respective LEDs are driven in the on mode. It may be that the photodiode's response is an output signal, such as a current signal that indicates a level (or amount) of the radiation (or light) received at (or detected by) the photodiode.

In order to determine a blood saturation value, the photodiode response is measured during the intervals of the cycle when the respective LEDs are driven in the on mode. The photodiode response may also be measured when the LEDs are operated in the off mode to provide metrics for calibration. The first and second LEDs are pulsed/driven, and the photodiode response is measured multiple times per cycle to enable fluctuations in the photodiode response arising from differences in volume of arterial blood present in the digit during the cycle (due to the patient's pulse rate) to be accounted for. The ratio of the photodiode's response to the red LED with respect to the photodiode's response to infrared LED is calculated, and the blood oxygenation value (e.g., SpO₂) is determined. For example, the blood oxygenation values may be calculated from the ratio of the photodiode response to red vs infrared light from look-up tables, which may be based on the Beer-Lambert law.

Additionally, the photodiode's response may be used to generate a plethysmography graphic, or tracing, that indicates the changes in volume of arterial blood observed during the cycle, and may be used to indicate a reliability of the blood oxygen saturation value determined by the pulse oximeter unit (or the pulse oximeter thereof).

The photodiode's 208 response 306, and/or values (e.g., calibrated values) generated therefrom, may be transmitted to the example receiver(s) described below.

It may be that the photodiode outputs a current (or current values) that is indicative of the radiation (or light) level received at (or detected by) the photodiode. For example, the photodiode may output a current signal e.g., current values as a function of time.

It may be that the current (or current signal) output by the photodiode is converted to a voltage (or voltage values, e.g., a voltage signal) before being transmitted to the example receiver(s) described below. For example, the current values output by the photodiode may be converted to voltage values by a wireless transmitter connected to the pulse oximeter unit, or by current to voltage conversion circuitry of the pulse oximeter unit. It may also be that the voltage values are buffered by the wireless transmitter to provide the respective first and second voltage data sets to be received by the receiver.

The voltage signal, or respective voltage data sets may be digitized and packetized for wireless transmission, and may be wirelessly transmitted to a receiver.

It may be that the receiver(s) described herein is (are) configured to receive data based on output from the pulse oximeter unit. For example, by way of the wireless transmitter(s) disclosed herein. It may be that the wireless transmitter(s) disclosed herein is (are) configured to receive an output from a pulse oximeter unit (e.g., over a wired connection) and, at least, wirelessly transmit data based on the output received from the pulse oximeter unit to the receiver(s) disclosed herein. For example, the wireless transmitter(s) may convert the pulse oximeter output to voltage values and buffer the voltage values into respective first and second data sets before transmitting the data to the receiver(s) disclosed herein.

The receiver(s) described herein may be configured such that the signal generation circuitry of the receiver(s) is configured to emulate the signal received by the patient monitor from a conventional pulse oximeter unit, over a wired connection, such that the receiver(s) described herein provide a wireless bridge to connect conventional (or legacy, i.e., intended for wired use) patient monitors and pulse oximeter units.

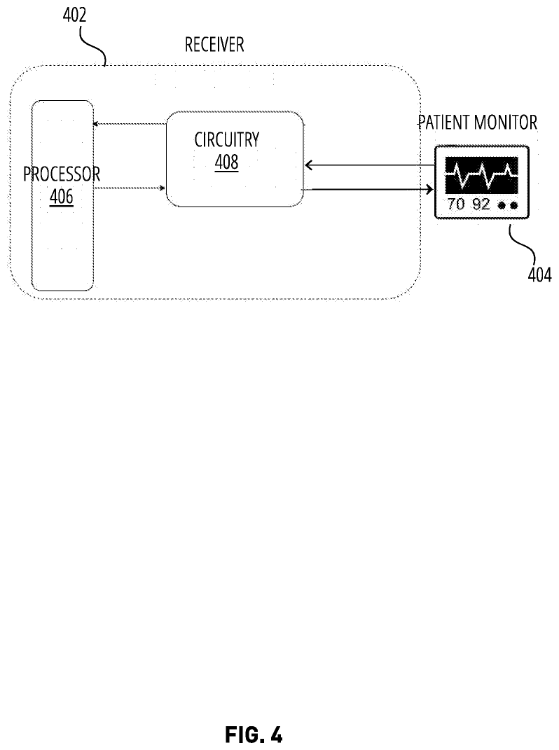

FIG. 4 shows a schematic diagram of functional blocks of an example receiver 402 connectable to a patient monitor 404. The receiver 402 may be the same as receiver 102. The receiver 402 comprises a processor 406 and circuitry 408 coupled to the processor 406. The circuitry 408 is configured to wirelessly receive data based on an output from a pulse oximeter unit (not shown). For example, the circuitry 408 may be configured to wirelessly receive the data by way of a wireless transmitter connected to the pulse oximeter unit. The pulse oximeter unit may comprise a pulse oximeter attached to a patient, as described above.

The data received by the receiver 402 (from the pulse oximeter unit) comprises a first data set comprising voltage values corresponding to a photodiode current sensed (or e.g., output) responsive to a first LED of the pulse oximeter unit. The data received by the receiver 402 also comprises a second data set comprising voltage values corresponding to a photodiode current sensed (or e.g., output) responsive to a second LED of the pulse oximeter unit.

For example, the photodiode current sensed responsive to the first or second LED of the pulse oximeter unit may be a current produced by the photodiode responsive to detecting the radiation (or light) from the respective first or second LED of the pulse oximeter unit, for example, after it has passed through the tissue of the patient that intervenes between the respective LED and the photodiode of the pulse oximeter unit.

It may be that the first data set comprises voltage values converted from the photodiode current sensed responsive to the first LED of the pulse oximeter unit. It may also be that the second data set comprises voltage values converted from the photodiode current sensed responsive to the second LED of the pulse oximeter unit.

For example, the photodiode currents sensed responsive to the respective first and second LEDs of the pulse oximeter unit may be converted to respective first and second voltage values by a transimpedance amplifier.

It may be that the photodiode of the pulse oximeter unit outputs a current signal comprising the current values output by the photodiode responsive to detecting the radiation (or light) from the first and second LEDs of the pulse oximeter unit in a single signal. For example, the current values corresponding to the sensed radiation for the first and second LEDs may be spaced apart at different timesteps in the signal. It may be that the voltage values are converted from the current values in the current signal, and may additionally be separated into the respective first and second data sets before or after the conversion. For example, the respective voltage values corresponding to the current sensed responsive to the first and second LEDs may be separated into the respective first and second data sets by buffering. For example, buffering of the voltage or current values at a wireless transmitter connected to the pulse oximeter unit.

The buffering may be based on a pre-determined frequency or duty cycle, for example, a duty-cycle pre-set in the wireless transmitter. For example, the buffering may comprise separating the data into the respective first and second data sets based on the frequency. It may be that such buffering is performed before transmission of the first and second data sets to the receiver(s) disclosed herein.

It may be that the first and second LEDs of the pulse oximeter unit are not driven by respective LED drive signals from the patient monitor. For example, it may be that when the pulse oximeter unit is not wired to the patient monitor the first and second LEDs of the pulse oximeter unit are driven independent of the patient monitor, for example at a frequency (or duty cycle) not indicated by the LED drive signals generated by the patient monitor. It may be that the first and second LEDs of the pulse oximeter unit are driven by respective LED drive signals determined by the pulse oximeter unit, or for example, at a pre-determined (e.g., pre-set) frequency. It may be that the patient monitor may sample (i.e., re-sample) the photodiode response data, e.g., the emulated signal received by the patient monitor, at the frequency corresponding to the respective LED drive signals generated by the patient monitor. In this way, in the absence of the wired connection between the patient monitor and the pulse oximeter unit, the patient monitor may sample (i.e., re-sample) the photodiode response data at the expected sampling rate of the patient monitor.

It may be that the first data set comprises voltage values corresponding to a photodiode current sensed responsive to a first LED of the pulse oximeter unit from which an ambient light signal has been subtracted. It may also be that second data set comprising voltage values corresponding to a photodiode current sensed responsive to a second LED of the pulse oximeter unit from which an (or the) ambient light signal has been subtracted.

For example, an ambient light signal may correspond to the photodiode response to ambient light passing through the portion of the patient's body intervening between the LEDs of the pulse oximeter unit and the photodiode, e.g., the tissue comprising the artery, such as the digit or earlobe. For example, the ambient light signal corresponds to the photodiode response when neither the first or second LED of the pulse oximeter of the pulse oximeter unit are driven in the on mode, e.g., when the tissue of the patient is not irradiated (or illuminated) by the first LED or the second LED.

It may be that the first data set comprises voltage values converted from the photodiode current output responsive to the first LED of the pulse oximeter unit from which the ambient light signal has been subtracted. It may also be that the second data set comprises voltage values converted from the photodiode current output responsive to the second LED of the pulse oximeter unit from which the ambient light signal has been subtracted.

For example, it may be that ambient light signal comprises current values (e.g., current values output by the photodiode in response to ambient light passing through the portion of the patient's body intervening between the LEDs of the pulse oximeter unit and the photodiode).

The current values of the ambient light signal may be subtracted from the photodiode current sensed from the first and second LEDs before the photodiode current values are converted to voltage values.

Alternatively, it may be that ambient light signal comprises voltage values (e.g., voltage values converted from current values output by the photodiode in response to ambient light passing through the portion of the patient's body intervening between the LEDs of the pulse oximeter unit and the photodiode). The voltage values of the ambient light signal may be subtracted from the voltage values corresponding to the photodiode current sensed (or output) responsive to the first and second LEDs after the photodiode current values are converted to voltage values. For example, before or after the separation (e.g., via buffering) of the different respective data sets, or before or after digitization of the respective data sets.

The receiver 402 is further configured to generate a signal based on the received data, wherein the signal is configured to emulate a wired connection input signal to the patient monitor. The receiver 402 outputs the generated signal to the patient monitor 404.

It may be that the receiver 402 is also configured to emulate a further effect on the patient monitor 404 that would be experienced by the patient monitor 404 as a result of a pulse oximeter unit being connected to the patient monitor 404 via a wired connection. For example, an effect in addition to the effect on the patient monitor 404 of receiving an input signal. In this way, the retrofitting of the receiver 402 to the patient monitor 404 may be improved.

For example, it may be that in a system with a wired connection between the patient monitor 404 and the pulse oximeter unit, the first and second LEDs of the pulse oximeter unit are driven by a signal from the patient monitor 404 communicated over the wired connection and/or are powered by the patient monitor over the wired connection. As a result, the first and second LEDs of the pulse oximeter unit may exert a load on the patient monitor 404. According to the wireless configuration of the receiver 402 for receiving the pulse oximeter data from the pulse oximeter unit, (i.e., there is no longer a wired connection between the patient monitor 404 and the pulse oximeter unit), the pulse oximeter unit no longer exerts a load on the patient monitor 404.

As described above, it may be that when the patient monitor is not wired to the pulse oximeter unit, the first and second LEDs of the pulse oximeter unit are driven independent of the respective LED drive signals generated by the patient monitor, for example, at a pre-determined, or pre-set frequency. It may also be that when the patient monitor is not wired to the pulse oximeter unit the first and second LEDs of the pulse oximeter unit are powered by a power supply (e.g., a battery) of the pulse oximeter unit. For example, the first and second LEDs of the pulse oximeter may be powered by the power supply of the pulse oximeter unit configured to power a processor or at least one other component of the pulse oximeter unit.

It may be that the receiver 402 may generate a load to emulate (e.g., to reproduce, or to cause the same, or a similar effect on the patient monitor 404) a load on the patient monitor 404 by the first and second LEDs of the pulse oximeter unit (e.g., if the pulse oximeter unit were connected to the patient monitor 404 via a wired connection, such as in a conventional patient monitoring system). The receiver 402 may be further configured to apply the emulated load to the patient monitor 404, for example by way of the circuitry 408. The emulated load applied to the patient monitor 404 by the receiver 402 may enable the patient monitor 404 to be operated with the receiver 402 while being blind to the removal of the wired connection to the pulse oximeter unit. For example, during use, the patient monitor 404 may not experience any deviation in operation when receiving the pulse oximeter data from the receiver 402 in comparison to receiving the pulse oximeter data via a wired connection. For example, it may be that the load applied to the patient monitor 404 operating with the receiver 402 and operating with a wired connection to the pulse oximeter unit may be indistinguishable by the patient monitor 404. It may be that, as a result of an indistinguishable change in load applied to the patient monitor 404, the patient monitor does not detect a fault during operation, such as a deviation from normal, or expected, signals transmitted over the connectors (or input/output lines) of the patient monitor. This may enable the receiver 402 to be retrofitted to the patient monitor 404 without requiring changes in operation to be made to account for the retrofitting, such as, for example, modifications to error or fault triggering or notifications.

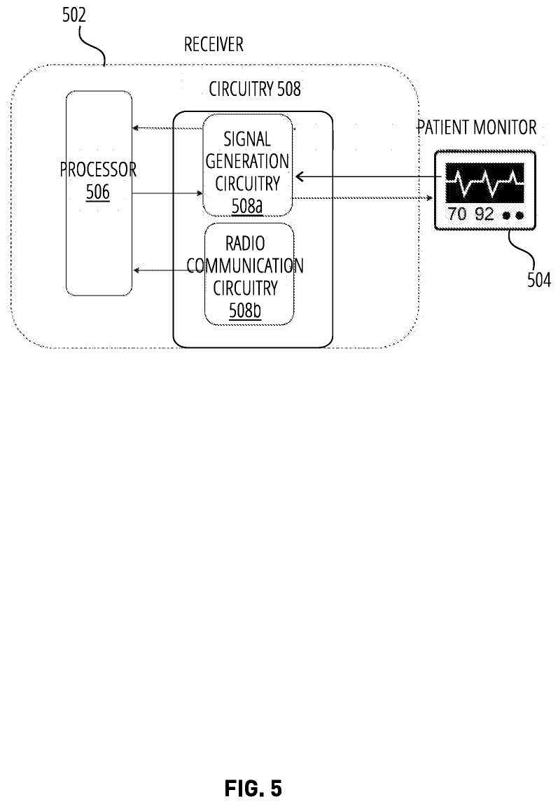

FIG. 5 shows a schematic diagram of functional blocks of an example receiver 502.

The circuitry 508 of receiver 502 comprises radio communication circuitry 508b configured to wirelessly receive a digitized and packetized signal comprising the data based on the output from the pulse oximeter unit and signal generation circuitry 508a to generate the signal to emulate the wired connection.

The circuitry 508, and for example, in particular radio communication circuitry 508b, is configured to wirelessly receive the data based on the output from the pulse oximeter unit. For example, the circuitry 508 may be configured to wirelessly receive the first data set comprising voltage values corresponding to a photodiode current sensed (or e.g., output) responsive to the first LED of the pulse oximeter unit (and optionally with an ambient light signal subtracted therefrom), and the second data set comprising voltage values corresponding to a photodiode current sensed (or e.g., output) responsive to the second LED of the pulse oximeter unit (and optionally with the ambient light signal subtracted therefrom).

It may be that the circuitry 508, for example radio communication circuitry 508b, is coupled to an antenna (not shown). The circuitry 508, for example radio communication circuitry 508b, may be configured to receive and process (e.g., at least de-modulate) the data that is wirelessly received from the pulse oximeter unit. It may be that the circuitry 508 comprises at least one of: a de-modulator, an amplifier, a mixer, and an oscillator, but the circuitry 508 is not so limited. For example, the circuitry 508 may further optionally comprise any one of a tuner and a filter etc.

The circuitry 508, for example radio communication circuitry 508b, may be configured to wirelessly receive the data via any wireless communication standard. For example, the circuitry 508 may be configured to receive the data from the pulse oximeter unit via any wireless communication standard for operation in a short-range communication network, such as a wireless local access network (WLAN), using e.g., Wi-Fi® (IEEE 802.11), or for any wireless communication standard for operation in a Wireless Personal Area Network (WPAN) or low-power personal area network (LPPAN), using, for example, any IEEE 802.15 wireless standard including Bluetooth®) (IEEE 802.15.1), or Zigbee® (IEEE 802.15.4), or any modifications or updates thereto. However, the circuitry 508 is not so limited and may alternatively be configured to receive the data wirelessly using other short-range communication networks or protocols, such as e.g., infrared data association (IrDA).

The circuitry 508 further comprises signal generation circuitry 508a configured to generate the signal to be output to the patient monitor 504. The signal generation circuitry 508a generates the signal configured to emulate (or e.g., reproduce at the patient monitor 504, or at an input thereof) a wired connection input signal to the patient monitor, for example the input signal received by the patient monitor 504 from the pulse oximeter unit when the pulse oximeter unit is coupled to the patient monitor 504 via a wired connection.

It may be that the signal generation circuitry 508a comprises switching circuitry controllable (i.e., configured to be controlled) to selectively provide the signal to the patient monitor 504 based on a respective one of the first and second data sets of voltage values received by the receiver 502 from the pulse oximeter unit.

For example, in a conventional wired patient monitoring system, the patient monitor 504 may drive the respective first and second LEDs of the pulse oximeter unit at different times, i.e., to irradiate or illuminate the tissue of the patient with the radiation emitted by the respective LEDs at different wavelengths (i.e., red optical light and infrared light) at different intervals, separated in time. In response to, for example, driving the first LED of the pulse oximeter unit in an on mode to irradiate the tissue of the patient, the patient monitor 504 may expect to receive a photodiode current sensed responsive to the first LED, and similarly for the second LED. However, when the wired connection between the patient monitor 504 and the pulse oximeter unit is removed, the direct relationship (i.e., causality) between which LED is driven by the patient monitor 504 and the LED photodiode response received by the patient monitor 504 may be negated, or at least weakened.

The signal generation circuitry 508a of the receiver 502 may provide the photodiode response to the respective LED of the pulse oximeter unit that is expected by the patient monitor 504, by controlling the switching circuitry to provide to the patient monitor 504 a generated signal based on the first or the second received data set corresponding to which of the first or second LED the patient monitor 504 is expecting to receive a photodiode signal with respect to. For example, during, or immediately following the interval in which the patient monitor 504 drives (i.e., provides a driving signal) to the first LED of the pulse oximeter unit (to operate the first LED in the on mode), the patient monitor 504 may expect to receive a photodiode response to the first LED. It may be that during or immediately following this interval, the signal generation circuitry 508a, or for example control logic thereof, may control the switching circuitry to provide to the patient monitor 504 the signal generated to emulate the photodiode response to the first LED provided to the patient monitor 504 over a wired connection. For example, by selecting the signal generated based on the first data set wirelessly received from the pulse oximeter unit. The same procedure, with respect to the second LED and second data set may be performed during or immediately following an interval during which the patient monitor 504 drives the second LED of the pulse oximeter unit in the on mode.

It may be that the signal generation circuitry 508a of the example receiver 502 (or, more particularly. control logic thereof) controls the switching circuitry responsive to receiving a first LED drive signal and/or a second LED drive signal (otherwise intended for the pulse oximeter unit via a wired connection) from the patient monitor 504.

For example, the receiver 502 may receive the first LED drive signal and/or the second LED drive signal over a wired connection to the patient monitor 504. It may be that the receiver 502 is connected to the patient monitor 504 via a (e.g., one or more) wired connection(s) that use (or e.g., re-use) the wired connection point(s) (e.g., ports) conventionally used to provide the output (i.e., the LED drive signal(s)) of the patient monitor 504 to the pulse oximeter unit.

It may be that the first LED and the second LED of the pulse oximeter unit are driven by separate (i.e., individual), respective, LED drive signals. It may be that the first and/or second LED drive signals received at the receiver 502 from the patient monitor 504 are respective first and second LED drive phase signals. For example, the respective drive signals may indicate whether the respective LEDs are driven in the on or off mode. For example, an LED drive phase signal may indicate as a function of time whether a respective LED is driven in the on or mode. In this respect, the respective LED drive phase signals comprise a binary indication of the mode of the respective LEDs.

It may also be that the processor 506 further controls the switching circuitry. For example, it may be that control logic of the signal generation circuitry 508a to control the switching circuitry to selectively provide respective signals to the patient monitor 504 is further controlled by the processor 506 responsive to an indication by the processor 506 of whether any of the LEDs of the pulse oximeter unit are driven in the on mode, or whether both the LEDs of the pulse oximeter unit are in the off mode.

It may be that the processor 506 is configured to control the switching circuitry to provide to the patient monitor 504 a dark level (or ambient light) voltage, corresponding to a photodiode response when neither the first nor second LED are irradiating the patient tissue during a time interval in which the patient monitor 504 does not drive either the first or the second LED in an on mode, i.e., during a time interval when both the first and second LEDs are in the off mode. For example, the dark level voltage may be a voltage that corresponds to the ambient light signal.

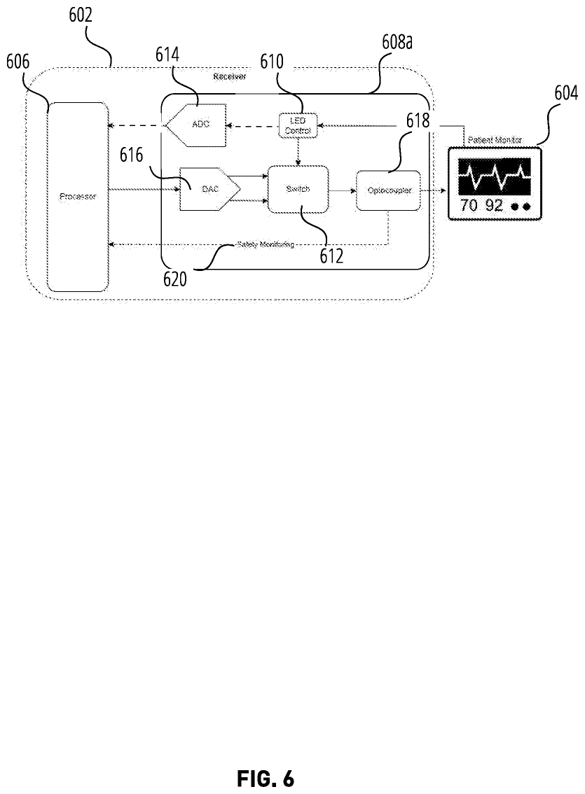

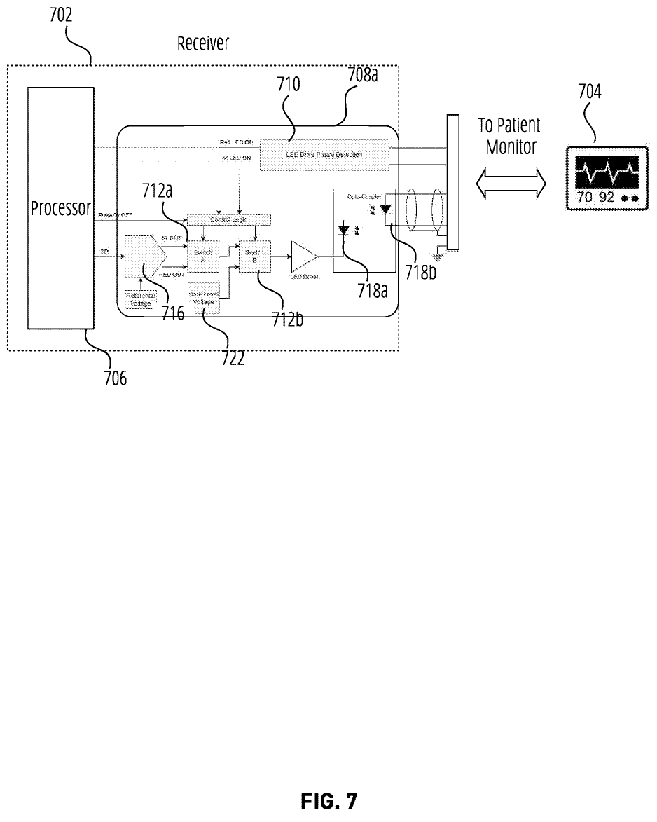

FIG. 6 shows a schematic circuit diagram of an example receiver 602. The receiver 602 may comprise the same functional blocks as receiver 102, receiver 402, or receiver 502.

Receiver 602 comprises switching circuitry 612 coupled to processor 606. The switching circuitry 612 is further coupled to an output from the patient monitor 604 comprising the first and second LED drive signals, for example first and second LED drive phase signals. As described above, at least the control logic (not shown in FIG. 6 for ease of illustration) (and optionally also the processor 606) controls the switching circuitry 612 to provide to patient monitor 604 a(n) (emulation) signal responsive to at least the first and/or second LED drive signals received by the receiver 602 from the patient monitor 604.

It may be that in addition to receiving the first and/or second LED drive phase signal(s), the receiver 602 also receives from the patient monitor 604 respective first and second LED drive current signals. It may be that the received first and/or second LED drive current signals indicate a current level, or current value, at which the respective LEDs of the pulse oximeter unit are to be driven.

It may be that the received first and/or second LED drive current signals are received as analogue signals from the patient monitor. Optionally, the circuitry 608a (e.g., the signal generation circuitry) at the receiver 602 may comprise an analogue to digital converter (ADC) 614 configured to convert the received LED drive current signals to digital signals. For example, the ADC may be coupled to the input from the patient monitor 604. The converted digital signals may be input to the processor 602.

It may be that the processor 602 is configured to, based on the received digitized LED drive current signals, control the signal generation circuitry 608a to generate a signal to be output to the patient 604 that emulates a wired connection input signal to the patient monitor that corresponds to a photodiode response to the first and second LEDs of the pulse oximeter unit being driven at the current level or value indicated in the respective LED drive current signals. For example, the processor 606 may scale the generated signal (e.g., the amplitude of the signal) to correspond to the photodiode response to the respective LEDs being driven at the indicated current. This may allow the receiver 602 to be adaptive to changes in the operation of the pulse oximeter unit controlled by the patient monitor, and may improve the retrofitting of the receiver 602 to the patient monitor 604.

The signal generation circuitry 608a of the receiver 602 comprises LED control circuitry 610 configured to receive the first and second LED drive phase signals (and optionally the first and second LED drive current signals) or to obtain (e.g., derive or determine) the respective LED drive phase (and optionally the drive current signals) from the input received from the patient monitor 604.

It may be that the LED control circuitry 610 comprises a first and a second LED, the same as, or similar to the respective first and second LEDs of the pulse oximeter unit. The respective first and second LEDs of the LED control circuitry 610 may be configured to be driven by the input signal (e.g., LED drive phase signals and optionally, LED drive current signals) received from the patient monitor 604. For example, the respective first and second LEDs of the LED control circuitry 610 may be configured to mirror or reproduce the operation of the respective first and second LEDs of the pulse oximeter unit.

It may be that the first and second LEDs of the LED control circuitry 610 emulate (or reproduce) the load on the patient monitor by the respective first and second LEDs of the pulse oximeter in a wired system, as disclosed herein.

The LED control circuitry 610 may further comprise LED signal detection circuitry (e.g., LED drive phase detection circuitry) to determine the respective first and second LED drive phase signals based on the operation of the respective first and second LEDs of the LED control circuitry 610. The LED signal detection circuitry may also comprise LED drive current detection circuitry to determine the respective first and second LED drive current signals based on the operation of the respective first and second LEDs of the LED control circuitry 610. It may be that the LED signal detection circuitry is configured to provide the first and second LED drive signals (e.g., LED drive phase) to the control logic of the receiver 602, an optionally to provide the respective LED drive current signals to the processor 606.

It may be that the LED drive phase signal for a respective LED is determined by monitoring the light level output from the corresponding LED of the LED control circuitry, rather than monitoring the current through said LED. For example, by monitoring the light level output by way of a photodiode. This may provide a non-invasive method for detecting the LED drive phase signal. For example, it may be that measuring the current through the LED may interfere with the patient monitor operation, which may introduce unexpected behaviour in the patient monitor. By monitoring the light level output from the respective LED of the receiver 602, this effect may be alleviated, or altogether avoided.

The circuitry 608a (e.g., the signal generation circuitry) of the receiver 602 comprises digital-to-analogue conversion, DAC, circuitry configured to convert the respective wirelessly received first and second voltage data sets from the pulse oximeter unit to respective first and second analogue voltage signals.

For example, it may be that the first and second voltage data sets are received from the pulse oximeter unit in a digitized signal. The DAC circuitry of the receiver 602 may convert the digitized signal to an analogue signal for further signal generation processing.

The signal generation circuitry 608a of the receiver 602 comprises optocoupler circuitry 618 configured to receive an analogue signal representing one of: voltage values corresponding to a photodiode current sensed (or e.g., output) responsive to the first LED of the pulse oximeter unit (and optionally with an ambient light signal subtracted therefrom); voltage values corresponding to a photodiode current sensed responsive to the second LED of the pulse oximeter unit (and optionally with an ambient light signal subtracted therefrom), or the dark level voltage values. It may be the analogue signals are selectively supplied to the optocoupler by the switching circuitry 612 controlled by at least control logic of the receiver 602.

The output of the optocoupler circuitry is coupled to an input (e.g., an input line or connector) of the patient monitor 604. More particularly, the output optocoupler circuitry is coupled to the input of the patient monitor 604 configured to receive the output from the pulse oximeter unit over a wired connection.

The optocoupler circuitry 618 may comprise an LED and at least one photodiode. The output of the at least one photodiode may be coupled to an input of the patient monitor 604. More particularly, the output of the photodiode may be coupled to the input of the patient monitor 604 configured to receive output from the pulse oximeter unit over a wired connection.

It may be that the output of the optocoupler circuitry 618 (e.g., the output of the photodiode) emulates the effect on the patient monitor 604 of the wired connection. For example, the output of the optocoupler (or e.g., the photodiode of the optocoupler circuitry) may supply a current to the patient monitor 604 input that is the same as the current that would be supplied to the input had the pulse oximeter unit been wired to the patient monitor 604.

Optionally, the optocoupler circuitry 618 may further comprise an additional, second, photodiode. The additional photodiode may be configured to provide an input to the processor 606. For example, the additional photodiode may input to the processor 606 a current feedback signal (or safety monitoring signal) 620. The feedback signal may enable the receiver 602 (via the processor 606) to respond to (e.g., emulate) changes in the current with which the patient monitor drives the LEDs of the pulse oximeter unit. This may improve compatibility with different models of patient monitor, and pulse oximeter units.

It may be that the feedback signal alternatively, or additionally, indicates to the processor a detected current that is to be output to the patient monitor, which may differ from a current intended, or expected, to be output to the patient monitor via the signal generation circuitry. For example, the feedback signal may allow the processor to detect and/or correct for faults in the operation of the signal generation circuitry, such as faults in a reference voltage supplied to the signal generation circuitry.

Additionally, or alternatively, the current feedback signal 620 may also be monitored by the processor 606 to avoid exceeding a threshold above which the circuitry of the receiver and/or the patient monitor may be harmed. For example, the feedback signal may indicate a drive level of the optocoupler from which it is received, which may be monitored by the processor 606 to ensure safe (i.e., not harmful) operation of the receiver 602 and/or the patient monitor.

Although the optocoupler circuitry 618 is described in some examples above as comprising at least one photodiode, it is to be understood that the optocoupler circuitry 618 is not so limited, and may comprise alternative photosensitive sensors, such as at least one phototransistor.

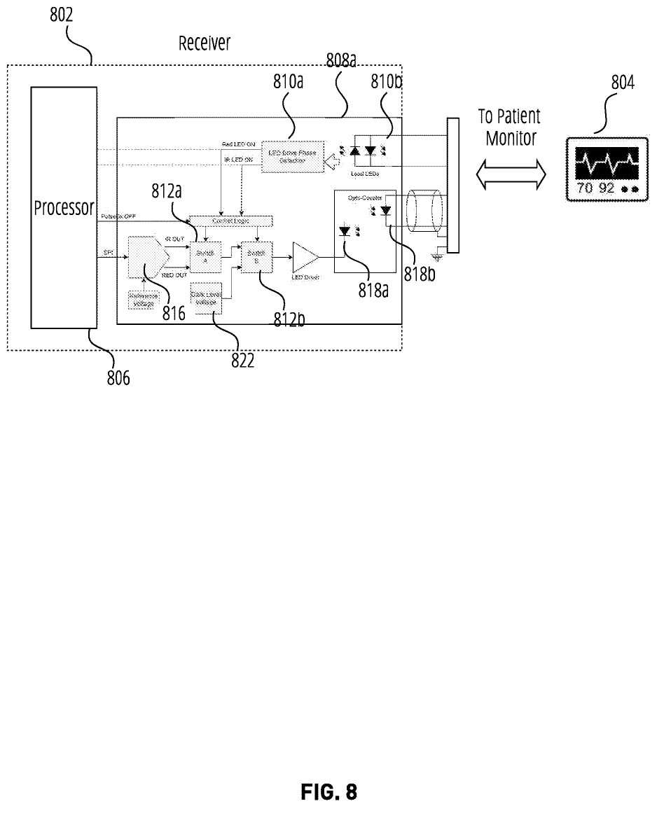

FIG. 7 shows a schematic circuit diagram of an example receiver 702. Receiver 702 may comprise all or some of the circuitry of receiver 602.

Receiver 702 includes switching circuitry coupled to processor 706 and an output from the control logic (which may be integrated with the LED control circuitry or otherwise be coupled to the LED control circuitry). The control logic is configured to receive first and second LED drive signals, for example first and second LED drive phase signals. As described above, at least the control logic (and optionally also the processor 706) controls the switching circuitry to provide to patient monitor 704 a(n) (emulation) signal responsive to at least the first and/or second LED drive signals received by the receiver 702 from the patient monitor 704.

For example, the control logic may be configured to select which of the first or second analogue voltage signals to provide to the optocoupler circuitry of the signal generation circuitry 708a of the receiver 702 at a given timestep based on the detected first and second LED drive signals.

In the example receiver 702, the switching circuitry comprises a first switch 712a coupled to the output of the DAC 716. The input of the DAC 716 is coupled to the output of the processor 706, and is configured to receive the digitized first and second voltage data sets received by the receiver 702 from the pulse oximeter unit. It may be that an additional input of the DAC 716 is coupled to a reference voltage supply, and is configured to receive the reference voltage and provide first and second analogue signals corresponding to the first and second voltage data sets, where the respective analogue signals are further based on the reference voltage supplied to the DAC 716.

The first switch 712a, receives the first and second analogue signals, corresponding to the respective first and second voltage data sets received by the receiver 702 from the pulse oximeter unit. The first switch 712a is coupled to an output of the control logic, and is configured to be controlled to deliver as output either the first or second analogue signal at a given timestep based on the control by the control logic. The control logic is coupled to the LED control circuitry and is configured to receive the first and second LED drive signals. The control logic is configured to select which of the first or second analogue voltage signals the first switch 712a is to provide to the optocoupler circuitry of the signal generation circuitry 708a of the receiver 702 at a given timestep based on the detected first and second LED drive signals. For example, at a given timestep, the control logic is configured to control the first switch 712a to provide as output the analogue signal corresponding to the LED that is being driven in the on mode. For example, at a timestep during which the first LED is being driven in the on mode, the control logic is configured to control the first switch 712a to provide as output the first analogue signal, which corresponds to the first voltage data set that comprises voltage values corresponding to a photodiode current sensed (or e.g., output) responsive to the first LED of the pulse oximeter unit (and optionally has an ambient light signal subtracted therefrom). Similarly, at a timestep during which the second LED is being driven in the on mode, the control logic is configured to control the first switch 712a to provide as output the second analogue signal, which corresponds to the second voltage data set that comprises voltage values corresponding to a photodiode current sensed (and optionally with the ambient light signal subtracted therefrom).

The switching circuitry of receiver 702 further comprises a second switch 712b. The second switch 712b is coupled to the output of the first switch 712a and is further coupled to an output from the control logic. An input of the control logic is also coupled to an output from the processor 706 that indicates whether either of the first or second LEDs of the pulse oximeter unit are being driven (e.g., during a given timestep or time interval), e.g., a pulse oximeter off indication. The control logic is configured, based on the pulse oximeter off indication, to control the second switch 712b to either provide as output the selected analogue signal output by the first switch 712a, or a dark level voltage. The dark level voltage may represent the voltage corresponding to the pulse oximeter unit photodiode response to an ambient light signal, i.e., when the tissue is not irradiated by the first or second LEDs of the pulse oximeter unit. The dark level voltage may, for example, be set to 0 Volts. For example, when the output from the processor 706, received by the control logic, indicates that the neither the first nor second LEDs are driven in the on mode during a given timestep, the switch 712b may be configured to output the dark level voltage. When the output from the processor 706, received by the control logic, indicates that the either the first or second LEDs are driven in the on mode during a given timestep, the switch 712b may be configured to output the analogue signal corresponding to the photodiode response to either the first or second LED, based on which LED the control logic determines is being driven in the on mode (based on the LED drive signals received by the control logic).

The output of the second switch 712b is coupled to the optocoupler circuitry.

As shown in FIG. 7, the optocoupler circuitry may comprise at least an LED 718a, and a photodiode 718b. However, the optocoupler circuitry is not so limited.

The photodiode 718b may be coupled to, or coupled to the output from (i.e., configured to receive incident radiation from), the LED 718a, to generate an output (e.g., a current) indicative of the output from the LED 718a.

As shown in FIG. 7, the output of the photodiode 718b may be coupled to at least one input line of the patient monitor 704. For example, the at least one input line of the patient monitor 704 that is configured to receive an output from the pulse oximeter unit over a wired connection.

The LED control circuitry of the example receiver 702 illustrated in FIG. 7 comprises LED drive phase detection circuitry 710. The LED drive phase detection circuitry 710 is coupled to an input (e.g., a wired input) from the patient monitor 704, and outputs a first LED drive phase signal and a second LED drive phase signal.

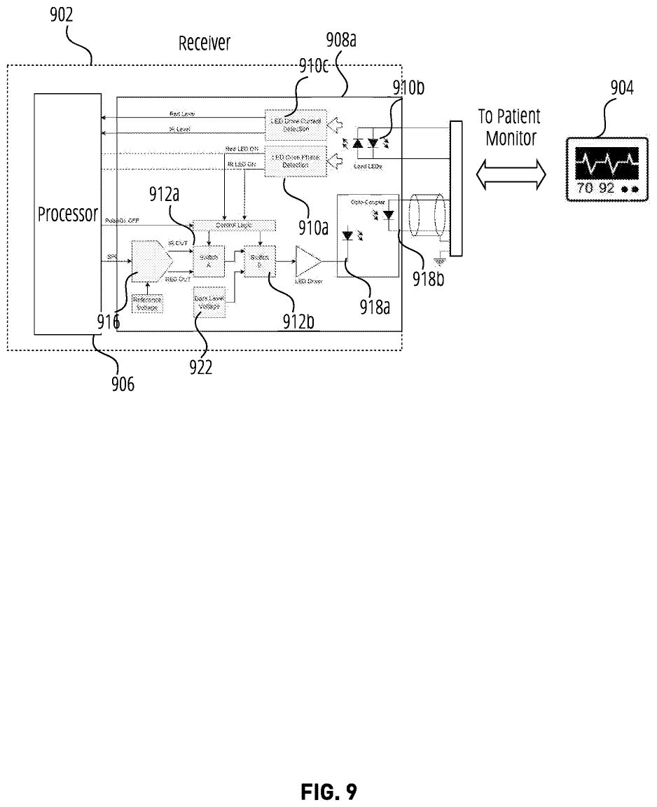

FIG. 8 shows a schematic circuit diagram of an example receiver 802. Receiver 802 may comprise some or all of the circuitry of receiver 602 or receiver 702.

As shown in FIG. 8, the LED control circuitry of the example receiver 802 further comprises first and second LEDs 810b, in addition to LED drive phase detection circuitry 810a.

It may be that the first and a second LEDs 810b are, the same as, or similar to the respective first and second LEDs of the pulse oximeter unit. The respective first and second LEDs 810b are configured to be driven by the input signal (e.g., LED drive phase signals and optionally, LED drive current signals, or combination thereof) received from the patient monitor 804. For example, the respective first and second LEDs 810b may be configured to mirror or reproduce the operation of the respective first and second LEDs of the pulse oximeter unit.

It may be that the first and second LEDs 810b emulate (or reproduce) the load on the patient monitor 804 by the respective first and second LEDs of the pulse oximeter in a wired system, as disclosed herein.

The LED drive phase detection circuitry 810a is coupled to, or respective to, the LEDs 810b (or output therefrom) to determine the respective first and second LED drive phase signals based on the operation of the respective first and second LEDs 810b.

It may be that the LED drive phase detection circuitry 810a is configured to monitor the light level output from the corresponding LEDs 810b, rather than monitoring the current through said LED 810b. For example, the LED drive phase detection circuitry 810a may at least comprise a photodiode. As described above, this may provide a non-invasive method for detecting the respective LED drive phase signals. For example, it may be that measuring the current through the respective LEDs 810b may interfere with the patient monitor operation, which may introduce unexpected behaviour in the patient monitor. By monitoring the light level output from the respective LEDs 810b of the receiver 802, this effect may be alleviated, or altogether avoided.

FIG. 9 shows a schematic circuit diagram of an example receiver 902. Receiver 902 may comprise some or all of the circuitry of receiver 602, receiver 702, or receiver 802.

As shown in FIG. 9, the LED control circuitry of the example receiver 902 further comprises LED drive current detection circuity 910c, in addition to LED drive phase detection circuitry 910a and first and second LEDs 910b.

The LED drive current detection circuitry 910c is coupled to, or respective to, the LEDs 910b (or output therefrom) to determine the respective first and second LED drive current signals based on the operation of the respective first and second LEDs 910b.

The LED drive current detection circuitry 910c is coupled to processor 906 to provide the respective first and second LED drive current signals to the processor 906.

As described above, it may be that the processor 902 is configured to, based on the received (digital) LED drive current signals, control the signal generation circuitry 908a to generate a signal to be output to the patient 904 that emulates a wired connection input signal to the patient monitor that corresponds to a photodiode response to the first and second LEDs of the pulse oximeter unit being driven at the current level or value indicated in the respective LED drive current signals. For example, the processor 906 may scale the generated signal (e.g., the amplitude of the signal) to correspond to the photodiode response to the respective LEDs being driven at the indicated current. This may allow the receiver 902 to be adaptive to changes in the operation of the pulse oximeter unit controlled by the patient monitor, and may improve the retrofitting of the receiver.

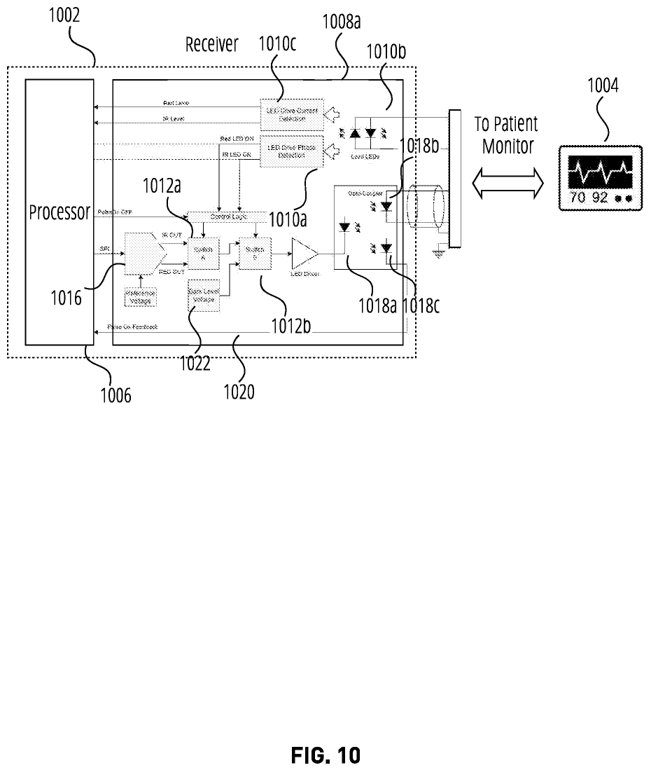

FIG. 10 shows a schematic circuit diagram of an example receiver 1002. Receiver 1002 may comprise some or all of the circuitry of receiver 602, receiver 702, receiver 802, or receiver 902.

As shown in FIG. 10, the optocoupler circuitry further comprises an additional (second) photodiode 1018c, in addition to the LED 1018a and the first photodiode 1018b.

The second photodiode 1018c is coupled to, or coupled to the output from (i.e., configured to receive incident radiation from), the LED 1018a, to generate an output (e.g., a current) indicative of the output from the LED 1018a. The photodiode 1018c is also coupled to the processor 1006, to input a current feedback signal to the processor 1006.

As described above, the feedback signal may enable the receiver 1002 (via the processor 1006) to respond to (e.g., emulate) changes in the current with which the patient monitor drives the LEDs of the pulse oximeter unit. This may improve compatibility with different models of patient monitor, and pulse oximeter units. It may be that the feedback signal is alternatively, or additionally, used to indicate to the processor a detected current to be output to the patient monitor, which may allow the processor to detect faults and/or correct faults in the operation of the signal generation circuitry.

Additionally, or alternatively, the current feedback signal may also be monitored by the processor 1006 to avoid exceeding a threshold above which the circuitry of the receiver and/or the patient monitor may be harmed. For example, the feedback signal may indicate a current level to be supplied to the patient monitor 1004, which may be monitored by the processor 1006 to ensure safe (i.e., not harmful) operation of the patient monitor 1004 and/or the receiver 1002.

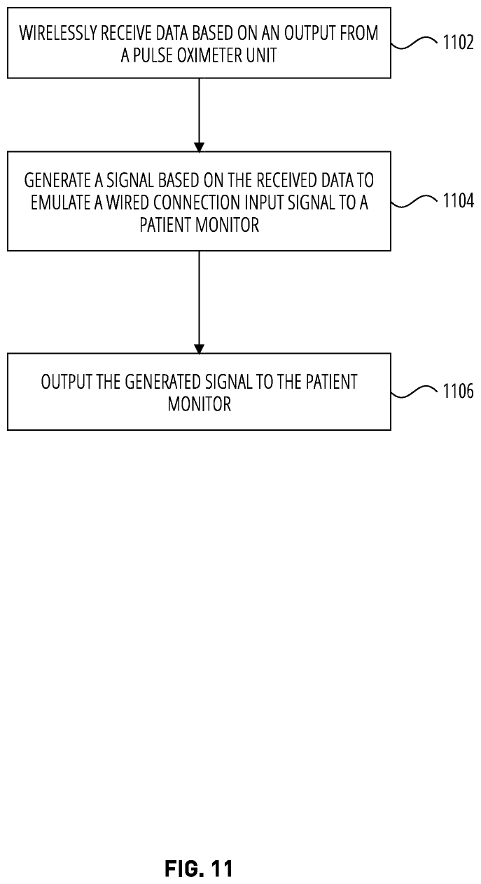

FIG. 11 is a flow chart illustrating an example method to be performed by a receiver connectable to a patient monitor.

In block 1102 the method comprises wirelessly receiving data based on an output from a pulse oximeter unit. As described above, the data comprises a first data set comprising voltage values corresponding to a photodiode current sensed responsive to a first LED of the pulse oximeter unit, and a second data set comprising voltage values corresponding to a photodiode current sensed responsive to a second LED of the pulse oximeter unit (and may optionally have an ambient light signal subtracted therefrom).

As described herein, the photodiode current sensed responsive to the first or second LED of the pulse oximeter unit may be a current produced by the photodiode responsive to detecting the radiation (or light) from the respective first or second LED of the pulse oximeter unit, for example, after it has passed through the tissue of the patient that intervenes between the respective LED and the photodiode of the pulse oximeter unit.

It may be that the first data set comprises voltage values converted from the photodiode current sensed responsive to the first LED of the pulse oximeter unit. It may also be that the second data set comprises voltage values converted from the photodiode current sensed responsive to the second LED of the pulse oximeter unit.

It may be that the first data set comprises voltage values corresponding to a photodiode current sensed responsive to a first LED of the pulse oximeter unit from which an ambient light signal has been subtracted. It may also be that second data set comprising voltage values corresponding to a photodiode current sensed responsive to a second LED of the pulse oximeter unit from which an (or the) ambient light signal has been subtracted.

It may be that the first data set comprises voltage values converted from the photodiode current output responsive to the first LED of the pulse oximeter unit from which the ambient light signal has been subtracted. It may also be that the second data set comprises voltage values converted from the photodiode current output responsive to the second LED of the pulse oximeter unit from which the ambient light signal has been subtracted.

In block 1104 the method comprises generating a signal based on the received data. The generated signal is configured to emulate a wired connection input signal to the patient monitor. For example, to allow retrofitting of the receiver to a conventional (i.e., intended for wired connection) patient monitor and pulse oximeter unit, for example, to provide a wireless bridge between the patient monitor and pulse oximeter unit to improve the robustness of patient vital sign monitoring and/or to enable freer movement of a patient being monitored. It may be that the signal is generated by signal generation circuitry of the receiver.

In block 1106 the method comprises outputting the generated signal to the patient monitor. It may be that the generated signal is communicated to the patient monitor via a wired connection between the receiver and the patient monitor. For example, the method may comprise outputting the generated signal over a wired connection between the receiver and the patient monitor.

It may be that the method also comprises generating a load to emulate a load on the patient monitor by the first and second LEDs of the pulse oximeter and applying the load to the patient monitor. For example, the load may be generated by signal generation circuitry of the receiver. It may be that the signal generation circuitry comprises first and second LEDs, similar to, or the same as, the first and second LEDs of the pulse oximeter unit. It may be that the method comprises driving the first and second LEDs of the signal generation circuitry of the receiver to generate a load of on the patient monitor that emulates the load on the patient monitor by the first and second LEDs of the pulse oximeter unit, when the pulse oximeter unit is wired to the patient monitor.

It may be that wirelessly receiving data from the pulse oximeter unit comprises at least one of, receiving a radio signal comprising the data (e.g., a carrier signal modulated with the data) via an antenna, and demodulating the received radio signal. It may be that the data is received using any short-range communication network and protocol for use therewith, as described above.

It may be that generating the signal based on the received data comprises selectively providing to the patient monitor, for example via switching circuitry of the receiver, a signal based on one of the received first data set or second data set. It may be that the selection of the signal to provide to the patient monitor is based on a received LED drive signal, indicating at a particular timestep which, if either, of the first or second LEDs of the pulse oximeter unit are being driven (e.g., in the on mode). It may be that the LED drive signal(s) is (are) received by the receiver from the patient monitor. It may be that the method performed by the receiver comprises deriving or detecting the LED drive signal(s).

It may be that selectively providing a signal to the patient monitor comprises controlling by at least control logic (and optionally the processor) of the receiver switching circuitry, to provide, at respective timesteps, a signal corresponding to a photodiode current sensed responsive to a first LED of the pulse oximeter unit to the patient monitor, or a signal corresponding to a photodiode current sensed responsive to a second LED of the pulse oximeter unit (or optionally a signal corresponding to an ambient light signal).

It may be that generating the signal based on the wirelessly received data comprises performing, for example, by a DAC of the signal generation circuitry, digital-to-analogue conversion of a signal to be input to the switching circuitry. For example, converting a wirelessly received digitized signal that is to be selectively provided to the patient monitor.

It may be that generating the signal based on the wirelessly received data comprises performing, for example by an ADC of the signal generation circuitry, analogue-to-digital conversion of an LED drive current signal received by (or for example derived or determined by) the receiver (from a received signal) from the patient monitor. For example, it may be that the method comprises controlling, by the processor of the receiver, the generated signal to emulate a wired connection input signal to the patient monitor that corresponds to a photodiode response to the first and second LEDs of the pulse oximeter unit being driven at the current level or value indicated in the respective LED drive current signals. For example, the method may comprise scaling, by the processor, the generated signal (e.g., the amplitude of the signal) to correspond to the photodiode response to the respective LEDs being driven at the indicated current. This may allow the receiver to be adaptive to changes in the operation of the pulse oximeter unit controlled by the patient monitor, and may improve the retrofitting of the receiver 602 to the patient monitor.

It may be that generating the signal based on the wirelessly received data and outputting the generated signal to the patient monitor comprises driving an optocoupler to provide the selected signal as input to the patient monitor. For example, the method may include driving an LED in an on or off mode based on the generated signal, and detecting by a photodiode the radiation (or light) emitted by the LED. It may be that the method also comprises supplying to an input line of the patient monitor the current output by the photodiode. For example, the current may be supplied to an input line of the patient monitor configured to receive a wired input from the pulse oximeter unit.

It may be that generating the signal based on the wirelessly received data comprises detecting by a second photodiode of the optocoupler circuitry of the receiver the radiation (or light) emitted by the LED of the optocoupler circuitry. For example, it may be that the method provides the second photodiode output as a feedback signal to the processor, i.e., the method comprises feeding back a current value corresponding to the level at which the LED of the optocoupler is being driven. This may enable the processor to monitor, e.g., for safe operation of at least one of the receiver and patient monitor, the drive level of the optocoupler (and e.g., thereby the signal generation circuitry and patient monitor input).

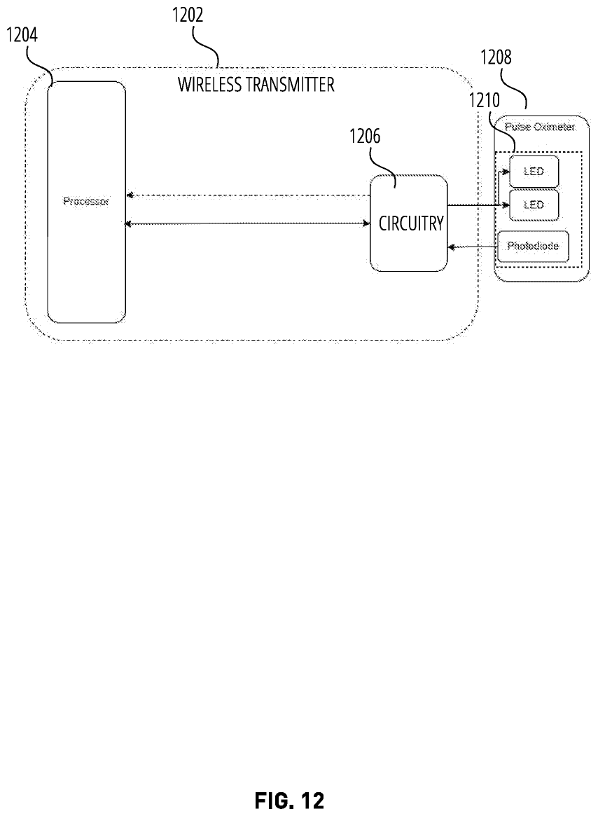

FIG. 12 shows a schematic diagram of functional blocks of an example transmitter connectable to a pulse oximeter unit.

As described above, the pulse oximeter unit 1208 comprises at least a pulse oximeter 1210.

The transmitter 1202 connectable to the pulse oximeter unit 1208, comprises a processor 1204. The transmitter 1202 further comprises circuitry 1206. The circuitry 1206 is coupled to the processor 1204, and is configured to receive a photodiode response signal from the pulse oximeter unit 1208. For example, the transmitter may be coupled, over a wired connection to the pulse oximeter unit.

The circuitry 1206 is configured to, based on a first LED drive signal and the received photodiode response signal, generate a first data set comprising voltage values corresponding to a photodiode current sensed responsive to the first LED of the pulse oximeter unit.

The circuitry 1206 is further configured to generate a second data set comprising voltage values corresponding to a photodiode current sensed responsive to the second LED of the pulse oximeter unit based on the received photodiode response signal and a second LED drive signal.

The circuitry 1206 is further configured to generate a digitized and packetized signal comprising the first and second data sets and wirelessly transmit the signal.

It may be that the respective first and second LED drive signals have a pre-determined, or for example pre-set, frequency or duty cycle. For example, it may be that respective first and second LED drive signals are generated by the processor 1204 based on a pre-determined frequency. In this way, the respective first and second LEDs of the pulse oximeter can be driven without a wired connection to the patient monitor.

It may be that the circuitry 1206 comprises at least one buffer configured to generate the respective data sets, i.e., separate out the photodiode current into the respective data sets.

It may be that the photodiode response signal comprises a photodiode current. The circuitry 1206 may further comprises current to voltage conversion circuity, such as a transimpedance amplifier, to convert the photodiode current output by the pulse oximeter unit into a voltage. It may be that the current to voltage conversion is performed before or after the buffering. For example, the output of the current to voltage conversion circuitry may be connected to an input of the at least one buffer, or an output of the at least one buffer may be connected to an input of the current to voltage conversion circuitry. Alternatively, it may be that the photodiode response signal received at the transmitter comprises a voltage signal (already) converted, for example by current to voltage conversion circuitry of the pulse oximeter, from the photodiode current output by the pulse oximeter.

It may be that the circuitry 1206 comprises ambient light signal subtraction circuitry to generate the respective first and second data sets from which the ambient light signal has been subtracted. It may be that the ambient light signal is subtracted prior to the current to voltage conversion and/or the buffering.

The circuitry 1206 is further configured to generate a digitized and packetized signal comprising the first and second data sets and to cause wireless transmission the signal, for example by way of an antenna (not shown).

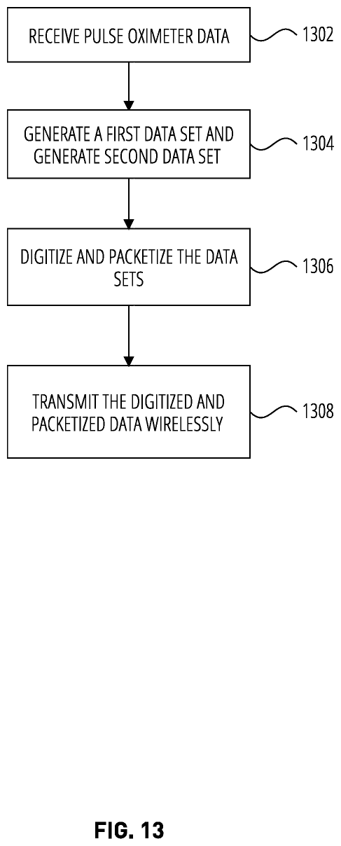

FIG. 13 is a flow chart illustrating an example method to be performed by a transmitter connectable to a pulse oximeter unit.

In block 1302 the method comprises receiving by the transmitter pulse oximeter data from the pulse oximeter unit.

It may be that the transmitter receives the pulse oximeter data from the pulse oximeter unit over a wired connection. For example, the transmitter may receive a photodiode response signal, such as a photodiode current, or voltage signal converted from the photodiode current, output by the pulse oximeter unit over a wired connection from the pulse oximeter unit.

It may be that the method comprises converting the received photodiode current to a voltage. Alternatively, the photodiode response signal received by the transmitter may already be a voltage signal.

In block 1304 the method comprises generating a first data set comprising voltage values corresponding to a photodiode current sensed responsive to the first LED of the pulse oximeter unit based on the received photodiode response signal and a first LED drive signal. The method further comprises generating a second data set comprising voltage values corresponding to a photodiode current sensed responsive to the second LED of the pulse oximeter unit based on the received photodiode response signal and a second LED drive signal.

In block 1306 the method comprises digitizing and packetizing the respective first and second data sets for wireless transmission. It may be that the digitization and packetization of the respective data sets may be performed by any well-known radio signal processing method and means, and may for example conform to any short-range wireless communication standard protocol requirements.

In block 1308 the method comprises wirelessly transmitting the digitized and packetized signal (e.g., to the receiver). The method may comprise wirelessly transmitting the signal via an antenna. The method may comprise wirelessly transmitting the signal using any short-range wireless communication standard protocols, as described above.

FIG. 14 shows a schematic diagram of functional blocks of an example system 1402. The system 1402 comprises a receiver 1404 connectable to a patient monitor 1408. Although receiver 1404 as illustrated correspond to receiver 602, it is not so limited may be any of the example receivers described above, for example with respect to FIGS. 1 and 4-10. The system 1402 further comprises a transmitter 1406 connectable to a pulse oximeter unit 1410. The transmitter may be any of the transmitters described above, for example with respect to FIG. 12.

The system 1402 may further comprise the patient monitor 1408. Alternatively, or additionally, the system may further comprise the pulse oximeter unit 1410.

The system 1402 may alternatively, or additionally comprise at least two electrodes (not shown) configured to provide an electro-cardiogram measurement and/or a temperature sensor (not shown). The system may further comprise am ECG unit and/or a temperature sensor unit (as will be described in more detail below).

The system may alternatively, or additionally comprise a transmitter configured to wirelessly transmit ECG data and/or temperature sensor data.

Example receivers configured to wirelessly receive data sensed by an electro-cardiogram (ECG) unit and a temperature sensor unit are described below. It may be that the data sensed by an the ECG unit (i.e., ECG data) and the data sensed by the temperature sensor unit (e.g., temperature sensor data) is received by the receiver from the respective ECG unit and temperature sensor unit, for example, by way of a wireless transmitter connected to the ECG unit and the temperature sensor unit.

A brief explanation of the basic operating principles of a temperature sensor unit is presented in order to aid understanding of the signals received and generated by the example receivers. A temperature sensor unit may be attached to a patient, i.e., to a body part thereof, to detect a skin temperature at the attachment site. The skin temperature of a patient, or body part thereof may indicate a vital sign of the patient. The temperature sensor unit may be attached to the patient, for example, by way of a clip or clamp, or via adhesion to the patient, for example using an adhesive applied to a pad on which the temperature sensor unit is mounted. However, any attachment means may be used. The temperature sensor unit comprises a temperature sensor, that may be configured to change an output in response to a change in temperature of, or at the sensor, for example a change in temperature detected, or experienced by a probe, or contact of the temperature sensor. For example, the temperature sensor may comprise a thermistor, or resistance thermometer, that has a resistance that is dependent on (i.e., changes with) temperature. The temperature sensor unit may output a signal, such as a voltage signal, that is dependent on the resistance of the temperature sensor. The patient monitor may use any well-known technique to measure the variable resistance of the temperature sensor based on the signal output by the temperature sensor. For example, the patient monitor may use a Wheatstone bridge, or any modification thereof. In this way the skin temperature of the patient may be monitored.

The voltage signal may be digitized and packetized for wireless transmission, and may be wirelessly transmitted to a receiver, for example by the wireless transmitter(s) disclosed herein. For example, the receivers disclosed herein configured to wirelessly receive data sensed from an electro-cardiogram (ECG) unit and a temperature sensor unit may be configured to wireless receive the respective data from the respective units by way of a wireless transmitter, such as the wireless transmitter(s) disclosed herein.

It may be that the receiver(s) described herein are configured to receive an output from the temperature sensor unit which has not been further processed with respect to the output from the temperature sensor unit that would be sent/transmitted over the wired connection, in a conventional system, with the exception of digitizing and packetizing the output for wireless transmission. This may improve the retrofitting, or flexibility to retrofitting the receiver(s) to conventional (i.e., configured/intended for wired communication) patient monitors and temperature sensor units.

It may be that when the temperature sensor unit is not wired to the patient monitor, the temperature sensor of the temperature sensor unit is not driven by the patient monitor. For example, it may be that when the temperature sensor unit is not wired to the patient monitor the temperature sensor is driven independent of the patient monitor, for example at a pre-determined (e.g., pre-set) voltage that may, for example, be provided by a power supply of the temperature sensor. Methods for accounting for the variation in the voltage at which the temperature sensor is driven are described below.

A brief explanation of the basic operating principles of an electro-cardiogram (ECG, or EKG) unit is also presented in order to aid understanding of the signals received and generated by the example receivers.

An ECG unit may comprise at least 2 electrodes configured to be attached to the patient body (e.g., chest) on opposing sides of the heart, in order to detect electrical signals indicative of the electrical activity of the patient's heart. The electrodes may be attached to the patient to provide electrical contact between the patient body and the electrodes, for example, by adhesion of the electrodes to the skin of the patient, or clips etc.

The ECG unit may be configured to measure an electrical potential (i.e., a voltage) between the at least 2 electrodes. By monitoring the voltage over time, the ECG unit may detect changes in the electrical activity of the patient's heart during respective cardiac cycles. During a normal (i.e., healthy) cardiac cycle the electrical signals from the heart (measured as a voltage) may follow a known pattern, or waveform, with recognisable components. For example, the measured ECG signal (voltage signal) may comprise a waveform, including, but not limited to P, QRS and T waveform features. The duration and shape of each waveform, or feature thereof, and the distances between different waveform features or characteristics (such as the peaks) may be used to indicate the health of the patient's heart, or deviations therefrom and is considered to be an indicator of a vital sign of the patient. Although the ECG unit is described above as having at least 2 electrodes, it may comprise more electrodes, for example, up to 10 electrodes to be attached to different portions of the patient body including limbs thereof.

The voltage signal(s) output by the ECG unit may be digitized and packetized for wireless transmission, and may be wirelessly transmitted to a receiver.

As described above with respect to the temperature sensor data and pulse oximeter data, it may be that the receiver(s) described herein are configured to receive an output from the ECG unit which has not been further processed with respect to the output from the ECG unit that would be sent/transmitted over the wired connection, in a conventional system, with the exception of digitizing and packetizing the output for wireless transmission. This may improve the retrofitting, or flexibility to retrofitting the receiver(s) to conventional (i.e., configured/intended for wired communication) patient monitors and ECG units.

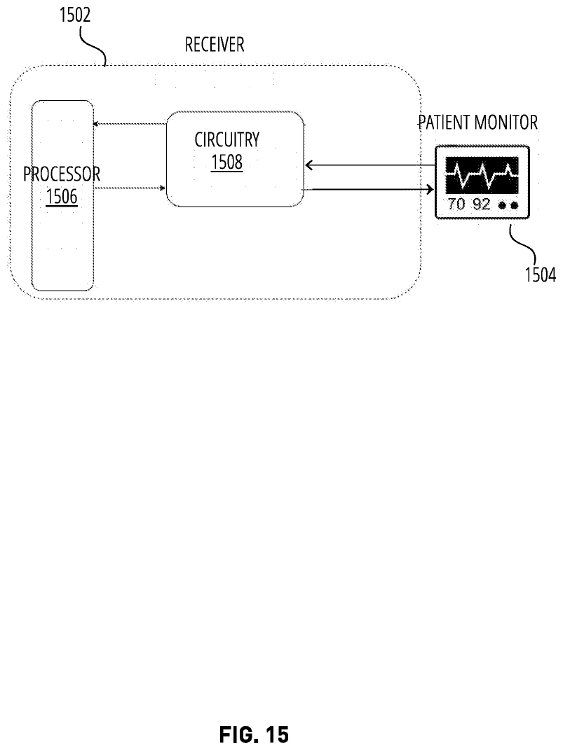

FIG. 15 shows a schematic diagram of functional blocks of an example receiver 1502 connectable to a patient monitor 1504. The patient monitor may be the same patient monitor, or the same as, or similar to any of the patient monitors described above, for example with respect to FIG. 1, and 4-10. The receiver 1502 comprises a processor 1506 and circuitry 1508 coupled to the processor 1506. The circuitry 1508 is configured to wirelessly receive ECG data sensed by an ECG unit (not shown) and to wirelessly receive temperature sensor data sensed by a temperature sensor unit (also not shown).

It may be that the ECG data comprises at least one voltage signal, i.e., it may comprise voltage values as a function of time. The ECG data may comprise at least one voltage signal representative of a difference in potential across two electrodes of the ECG unit. Alternatively, it may be that the ECG data comprises more than one voltage signal, for example two voltage signals. It may be that the relative offset or difference between the voltage signals (e.g., at respective timesteps) represents the difference in potential across two electrodes of the ECG unit. For example, at least one of the voltage signals may represent a reference voltage.

It may be that the temperature sensor data comprises a voltage signal. The temperature sensor data may comprise a voltage signal based on a resistance controlled by the temperature sensor.

The receiver 1502 is further configured to generate at least a first signal based on the received ECG data, wherein the at least first signal is configured to emulate first and second wired connection input signals received by the patient monitor. For example, the at least first signal is configured to produce the same effect at the patient monitor as the first and second wired connection inputs received by the patient monitor, e.g., in a conventional wired system. It may be that the at least first signal is a voltage signal. The at least first voltage signal may represent (or correspond to) the potential difference across the electrodes of the ECG unit.

It may be that the receiver 1502 is configured to generate more than one signal to emulate the effect on the patient monitor by the first and second wired connection inputs received by the patient monitor from the ECG unit over a wired connection. For example, the receiver 1502 may be configured to generate at least two signals e.g., a first and a second signal. It may be that the respective generated first and second signals of the at least two signals are first and second voltage signals. It may be that the relative offset or difference between at least two signals, e.g., the first and second voltage signals, represents, or corresponds to, the potential difference across the electrodes of the patient monitor. For example, it may be that at least one of the at least two, i.e., first or second, generated signals is, or represents, a reference voltage.

It may be that the at least first generated signal is configured to generate a voltage signal across first and second inputs (e.g., input lines) of the patient monitor that are configured to receive the wired connection input from the ECG unit that emulates (or reproduces) the voltage signal measured by the ECG unit, e.g., across the at least 2 electrodes of the ECG unit. For example, it may be that the receiver 1502 is configured to provide the first generated signal to a first input line of the patient monitor, and e.g., a reference voltage (such as a fixed, constant voltage e.g., at 0V) to a second input line of the patient monitor. Alternatively, the receiver 1502 may be configured to provide a first component of the first generated signal to a first input line of the patient monitor and a second component of the first generated signal to a second input line of the patient monitor. For example, it may be that the first and second component of the first generated signal are configured to generate a voltage signal across first and second inputs (e.g., input lines) of the patient monitor that emulates (or reproduces) the voltage signal measured by the ECG unit, e.g., across the at least 2 electrodes of the ECG unit.

Alternatively, it may be that the two or more (i.e., first and second) generated signals are configured to generate a voltage signal across first and second inputs (e.g., input lines) of the patient monitor that emulates (or reproduces) the voltage signal measured by the ECG unit, e.g., across the at least 2 electrodes of the ECG unit. For example, the receiver 1502 may be configured to output the first generated signal to a first input line of the patient monitor and to output the second generated signal to a second input line of the patient monitor (where the first and second input lines of the patient monitor are configured to receive outputs via a wired connection from the ECG unit. For example, it may be that the second signal is e.g., a time varying reference voltage.

The receiver 1502 is further configured to generate an additional signal (e.g., a second signal if only a first signal is generated with respect to the ECG unit data, or a third signal if two signals are generated with respect to the ECG unit data) based on the temperature sensor data. The additional (e.g., second or third) signal is configured to emulate a wired connection input signal received by the patient monitor from the temperature sensor unit.

The additional (e.g., second or third) signal may be a current signal (i.e., current values as a function of time). For example, the patient monitor may be configured such that a current is drawn from the patient monitor via a wired connection with the temperature sensor unit. It may be that the additional (e.g., second or third) signal is configured to emulate, or reproduce, the current drawn from the patient monitor over a wired connection from the temperature sensor. It may be that the patient monitor is configured to interpret the drawn current to determine a resistance corresponding to the resistance of the temperature sensor of the temperature sensor unit. For example, using a Wheatstone bridge, or modifications thereof. It may be that the patient monitor is configured to determine a temperature measurement from the derived resistance. The determined temperature may be presented, by the patient monitor, as an indication of a vital sign. It may be that the additional (e.g., second or third) signal, which may be a current signal, is configured to emulate or reproduce the temperature determined by the patient monitor from a current received over a wired connection.

The receiver 1502 outputs the generated signals to the patient monitor 1504. For example, the receiver 1502 may output the at least first (and optionally second) generated signal(s) on respective input lines of the receiver configured to receive wired connections from the ECG unit, and may output the additional (e.g., second or third) signal on an input line of the patient monitor configured to receive a wired connection from the temperature sensor unit.

It is to be understood, that while the respective signals are described herein as first, additional, and e.g., second and third signals, this is not to be construed as limiting, and that the signals described herein can be received, generated and output in any order. It is also to be understood that further signals may be received, generated and output. For example, further signals may be received, generated and output to reflect the number of electrodes, or electrode pairs of the ECG unit.

It may be that the receiver 1502 is also configured to emulate a further effect on the patient monitor 1504 that would be experienced by the patient monitor 1504 as a result of the temperature sensor unit and/or the ECG unit being connected to the patient monitor 1504 via a wired connection. For example, an effect in addition to the effect on the patient monitor 1504 of receiving the respective input signals. In this way, the retrofitting of the receiver 1502 to the patient monitor 1504 may be improved.

For example, it may be that the receiver 1502 is configured to emulate, or reproduce, a lead-off event at the ECG. During conventional operation, with wired connections, the patient monitor may be in direct communication (e.g., electrical communication) with the electrodes of the ECG unit and may be able to detect a lead-off event, i.e., when a lead or wire connecting a, or the, electrode(s) of the ECG unit to the patient monitor are detached or the communication is interrupted. As the direct communication link is broken with the introduction of the wireless transmission and reception of the data, the patient monitor may not otherwise be aware of such an event. The emulation, or reproduction, of the effects of this event at the patient monitor improves the retrofitting of the receiver to the patient monitor as it maintains the functionality of a wired system.

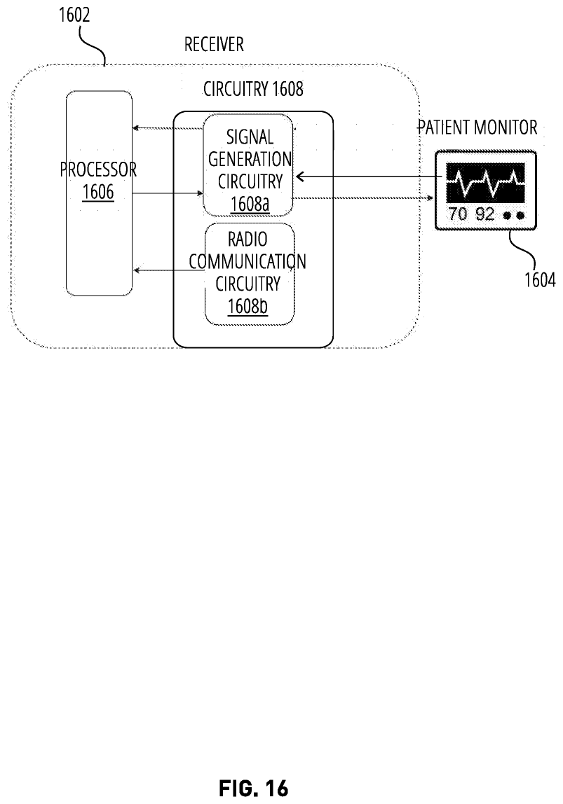

FIG. 16 shows a schematic circuit diagram of an example receiver 1602. The receiver 602 may comprise the same functional blocks as receiver 1502.

The circuitry 1608, and for example, in particular radio communication circuitry 1608b, is configured to wirelessly receive the data sensed by the temperature sensor unit and the ECG unit.

It may be that ECG data and the temperature sensor data are received by the receiver 1602 in the same radio signal. For example, the receiver 1602 may be configured to wirelessly receive the ECG data (e.g., a voltage signal) and the temperature sensor data (e.g., a voltage signal) by wirelessly receiving at least digitized and packetized ECG and temperature sensor data. It may be that the digitized ECG and temperature sensor data are packetized in respective packets. It may also be that each packet has a corresponding header. For example, a header indicating which data (e.g., which type of data: temperature or ECG, is in the corresponding packet).

However, the receiver is not so limited and may be configured to receive the ECG and temperature data via any well-known signalling procedure.

It may be that the circuitry 1608, for example radio communication circuitry 1608b, is coupled to an antenna (not shown). The circuitry 1608, for example radio communication circuitry 1608b, may be configured to receive and process (e.g., de-modulate) the data that is wirelessly received, e.g., from the temperature sensor and ECG sensor units by way of a wireless transmitter connected thereto. It may be that the radio communication circuitry 1608b is the radio communication circuitry 508b, or is the same, or similar to radio communication circuitry 508b described above. For example, it may be that the circuitry 1608 comprises at least one of: a de-modulator, an amplifier, a mixer, and an oscillator, but the circuitry 1608 is not so limited. For example, the circuitry 1608 may further optionally comprise any one of a tuner and a filter etc.

The circuitry 1608, for example radio communication circuitry 1608b, may be configured to wirelessly receive the data via any wireless communication standard. For example, the circuitry 1608 may be configured to receive the data from the temperature sensor unit and/or the ECG unit via any wireless communication standard for operation in a short-range communication network, such as a wireless local access network (WLAN), using e.g., Wi-Fi® (IEEE 802.11), or for any wireless communication standard for operation with via a Wireless Personal Area Network (WPAN) or low-power personal area network (LPPAN), using, for example, any IEEE 802.15 wireless standard including Bluetooth® (IEEE 802.15.1), or Zigbee® (IEEE 802.15,4), or any modifications or updates thereto. However, the circuitry 1608 is not so limited and may alternatively be configured to receive the data wirelessly using other short-range communication networks, such as e.g., infrared data association (IrDA).

The circuitry 1608 further comprises signal generation circuitry 1608a configured to generate the signals to be output to the patient monitor 1604. The signal generation circuitry 1608a generates the at least first (and optionally second) signal(s) configured to emulate (or e.g., reproduce at the patient monitor 1604, or at an input thereof) a wired connection input signal to the patient monitor, for example the input signal received by the patient monitor 1604 from the ECG unit when the ECG unit is coupled to the patient monitor 1604 via a wired connection.

The signal generation circuitry 1608a also generates the additional (e.g., second or third) signal configured to emulate (or e.g., reproduce at the patient monitor 1604, or at an input thereof) a wired connection input signal to the patient monitor, for example the input signal received by the patient monitor 1604 from the temperature sensor unit when the temperature sensor unit is coupled to the patient monitor 1604 via a wired connection.

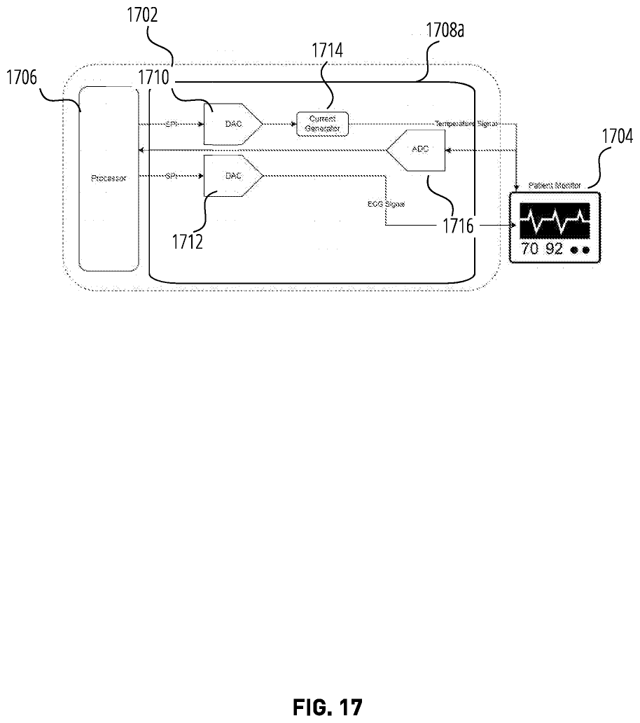

FIG. 17 shows a schematic circuit diagram of an example receiver 1702. The receiver 1702 may comprise the same functional blocks as receiver 1502, or receiver 1602.

The receiver 1702 comprises processor 1706 and circuitry (e.g., signal generation circuitry) 1708a.

The signal generation circuitry 1708a is configured to generate the at least first signal based on the received ECG data, wherein the at least first signal is configured to emulate first and second wired connection input signals received by the patient monitor. For example, the at least first signal is configured to produce the same effect at the patient monitor as the first and second wired connection inputs received by the patient monitor, e.g., in a conventional wired system.

Although FIG. 17 illustrates the signal generation circuitry 1708a generating and outputting a first signal, i.e., only a first signal, to the patient monitor, it is to be understood that the signal generation circuitry 1708a may generate first and second signals, or any plurality of signals, to be input to respective first and second input lines of the patient monitor to emulate the effect at the patient monitor of first and second wired connection inputs received by the patient monitor, e.g., in a conventional wired system.

The signal generation circuitry 1708a of receiver 1702 is also configured to generate the additional signal (i.e., the temperature signal) to emulate the wired connection input signals received by the patient monitor from the temperature sensor data.

The signal generation circuitry 1708a is configured to output the generated ECG signal(s) and generated temperature signal to the patient monitor, for example to input lines of the patient monitor configured to receive the corresponding wired connection inputs from the ECG unit and temperature sensor unit.

The signal generation circuitry 1708a is configured to receive the digitized ECG data and temperature sensor data from the processor 1706. It may be that the processor 1706 is configured to receive the digitized (and optionally still packetized) data from radio communication circuitry of the receiver, as described above with respect to FIG. 16. For example, the processor 1706 may be coupled to an output from the radio communication circuitry, which may be coupled to an antenna and be configured to at least demodulate the received radio signal(s), as described above with respect to FIG. 16. An output, or more than one output, of the processor may be coupled to the signal generation circuitry 1708a.

The signal generation circuitry 1708a comprises a DAC 1710 to convert the digitized temperature sensor data received from the processor 1706 to an analogue signal. For example, to convert a digitized voltage signal based on a resistance controlled by the temperature sensor to an analogue voltage signal.

The output of the DAC 1710 is coupled to a current generator 1714. It may be that in a conventional wired system, the patient monitor drives the temperature sensor unit at a given voltage via the wired connection. It may be that the change in the resistance of the temperature sensor (e.g., thermistor) causes the temperature sensor to change the current drawn from the patient monitor, and the patient monitor may infer or determine the resistance, and therefore, the detected temperature, based on the current (or changes in the current) drawn by the temperature sensor over the wired connection.

In order to emulate this effect with the receiver 1702, the current generator 1714 is configured to, based on the wirelessly received (analogue) voltage signal (that is based on, or indicates, a resistance controlled by the temperature sensor) draw a current from the patient monitor that causes the signal generation circuit pathway (e.g., the pathway between the processor and the patient monitor input configured to receive the wired connection from the temperature sensor unit, or a portion thereof) to have an impedance that emulates, or reproduces, the resistance of the temperature sensor of the temperature sensor unit. It may also be that the voltage across the signal generation circuitry pathway may not be known, or may be variable. In this case, it may be that the current to be drawn from the patient monitor to cause the impedance of the temperature sensor to be emulated, is based on the sensed voltage across the signal generation circuitry pathway. For example, the current drawn from the patient monitor may be set by a control loop.

The signal generation circuitry 1708a also comprises a DAC 1712 to convert the digitized temperature sensor data received from the processor 1706 to an analogue signal. For example, to convert a digitized voltage signal (that is based on, or indicates, a resistance controlled by the temperature sensor) to an analogue voltage signal.

The output of the DAC 1712 is coupled to the patient monitor, for example, by way of an input line of the patient monitor configured to receive the wired connection from the temperature sensor unit.

The signal generation circuitry 1708a may also be configured to provide a feedback signal (e.g., a feedback voltage signal) to the processor 1706. For example, to indicate the voltage at which the patient monitor drives the temperature sensor (or would drive the temperature sensor if wired to the temperature sensor unit). Based on the voltage at which the patient monitor drives (or would drive) the temperature sensor unit, the processor may be configured to dynamically adapt the current drawn by the signal generation circuitry, for example to account for, and e.g., emulate, variations in the voltage at which the patient monitor drives the temperature sensor unit. It may be that this feedback process improves the retrofitting of the receiver to the patient monitor and/or the temperature sensor unit.

Additionally, or alternatively, the signal generation circuitry 1708a may be configured to provide a feedback signal (e.g., a feedback current signal) to the processor to enable the processor to account for changes between an intended current to be drawn from the patient monitor, and an actual current drawn from the patient monitor. For example, to control or correct (e.g., in a control loop) the current drawn from the patient monitor, for example, if the current modulation/adjustment is performed non-linearly (e.g., by a non-linear transistor etc.). It may be that this feedback, or control loop, improves operation of the receiver to changes in impedance based on a change in temperature sensed by the temperature sensor.

It may be that the signal detection circuitry 1708a also comprises an ADC 1716 disposed between the patient monitor 1704 and the processor 1706 to convert the feedback analogue (voltage) signal from the patient monitor 1704 into a digital signal suitable to be processed by the processor 1706.

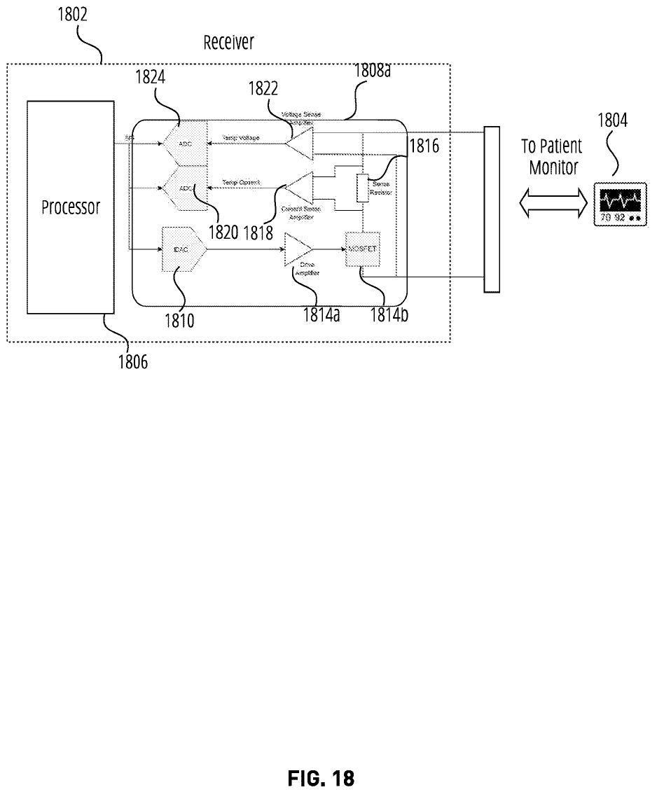

FIG. 18 shows a schematic circuit diagram of an example receiver 1802. The receiver 1802 may comprise all or some of the circuitry of receiver 1702.

The signal generation circuitry 1808a of receiver 1802 illustrated in FIG. 18 is configured to generate the signal configured to emulate the wired connection input signal received by the patient monitor from the temperature sensor unit. It may be that the signal generation circuitry 1808a of receiver 1802 forms part of, or all of, the signal generation circuitry 1708a or 1608a. For example, the current generator 1714 of FIG. 17 may comprise the current sensing circuitry and voltage sensing circuitry of FIG. 18.

The signal generation circuitry 1808a comprises DAC 1810 coupled to receive the voltage signal from the processor 1806 and draw a corresponding current from the patient monitor by way of drive amplifier 1814s and transistor (e.g., MOSFET) 1814b.

The signal generation circuitry 1808a also comprises current sensing circuitry configured to sense a current in the signal generation circuitry pathway (e.g., between the processor and a connector of the patient monitor 1804 configured to receive the wired connection input signal from the temperature sensor unit, or a portion thereof), and to provide the sensed current to the processor 1806.

It may be that, as illustrated in FIG. 18, the current sensing circuitry comprises at least sense resistor 1816 coupled to the signal detection circuitry pathway drawing current from the patient monitor 1804. The sense resistor 1816 may be coupled to current sense amplifier 1818. The output of current sense amplifier 1818 may be coupled to ADC 1820, which is configured to convert the sensed analogue current signal to a digital signal suitable of being processed by processor 1806. The sensed current signal is to indicate to the processor the actual current being drawn from the patient monitor 1804 by the signal generation circuitry 1808a, or at least the temperature signal emulation component thereof. It may be that the current sense circuitry forma part of a control loop to account for the non-linear nature of transistor 1814b of the signal generation circuitry 1808a.

The signal generation circuitry 1808a also comprises voltage sensing circuitry configured to sense a voltage across the signal generation circuit pathway (e.g., between the processor and the connector of the patient monitor configured to receive the wired input from the temperature sensor unit, or a portion thereof) and to provide the sensed voltage to the processor 1806.

It may be that the voltage sensing circuity, as illustrated in FIG. 18, comprises at least voltage sense amplifier 1822 and ADC 1824. The ADC is coupled to the output of the voltage sense amplifier 1822 to provide a digital voltage signal to the processor 1806.

The processor 1806 is configured to calculate, based on the sensed voltage and the wirelessly received temperature sensor data, a current to be drawn from the patient monitor 1804, wherein the current to be drawn causes the signal generation circuit pathway (e.g., between the processor and the patient monitor input line configured to receive the temperature sensor data over a wired connection, or a portion thereof) to have an impedance that emulates the resistance of the temperature sensor of the temperature sensor unit.

The processor 1806 is configured to adjust, based on the sensed current, the current drawn from the patient monitor to draw the calculated current, for example, to adjust the current drawn from the patient monitor to approximately, or substantially match, or equate to, the calculated current.

It is be understood that the current sensing circuitry and voltage sensing circuitry shown in FIG. 18 is an example for illustrative purposes the current sensing circuitry and voltage sensing circuitry is not so limited.

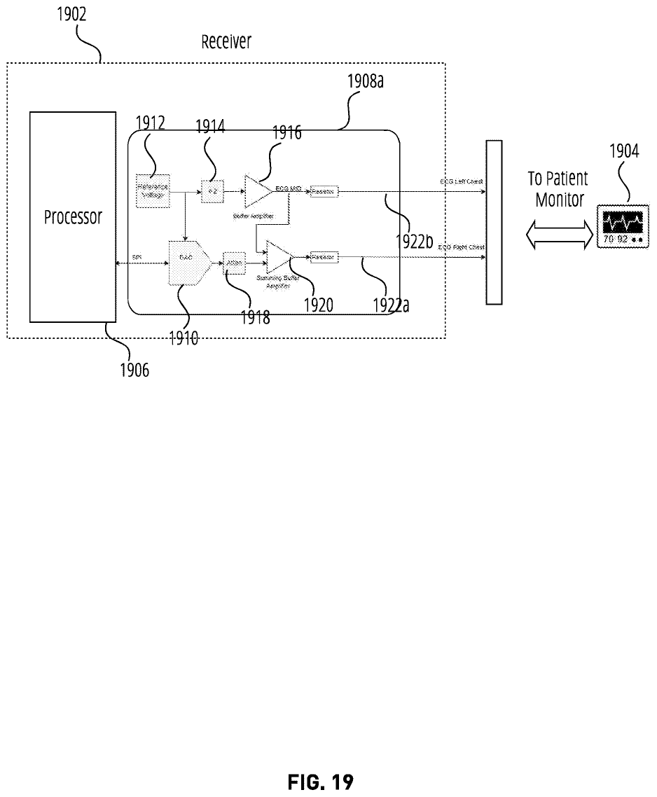

FIG. 19 shows a schematic circuit diagram of an example receiver 1902. The receiver 1902 may comprise all or some of the circuitry of receiver 1702 or receiver 1802.

The signal generation circuitry 1908a of receiver 1902 illustrated in FIG. 19 is configured to generate the at least one (first) signal configured to emulate the wired connection input signal received by the patient monitor from the ECG unit. It may be that the signal generation circuitry 1908a of receiver 1802 forms part of the signal generation circuitry 1708a or 1608a. For example, it may be that the receiver 1902 further comprises the signal generation circuitry 1808a illustrated in FIG. 18.

The signal generation circuitry 1908a comprises reference voltage signal generation circuitry configured to generate a constant reference voltage signal at a given reference voltage.

It may be that, as illustrated in FIG. 19, the reference voltage signal generation circuitry comprises, or is coupled to a reference voltage supply 1912. The reference voltage supply 1912 may provide a reference voltage to the voltage signal generation circuitry of (approximately) 2.5 Volts. The reference voltage supply 1912 may provide the same reference voltage to the voltage scaling circuitry and the voltage signal generation circuitry, e.g., 2.5 Volts.

The reference voltage supplied by the supply 1912 may be divided, for example by voltage divider 1914. It may be that voltage divider 1914 may be a resistor voltage divider, but it is not so limited. Voltage divider 1914 may output a voltage corresponding to the reference voltage reduced to (approximately) half of the voltage supplied by supply 1912, e.g., (approximately) 1.25 Volts.

It may be that the output of the voltage divider 1914 is buffered, for example by buffer amplifier 1916 coupled to the output of the voltage divider 1914. The buffered output voltage may be provided as output to the patient monitor. It may be that the output of the buffer amplifier 1916 is also provided to a summing buffer amplifier 1920 of voltage scaling circuitry of the receiver 1902.

The signal generation circuitry 1908a also comprises voltage scaling circuitry configured to scale a voltage signal based on the received ECG data such that the scaled voltage signal is centred around a given reference voltage.

It may be that, as illustrated in FIG. 19, the voltage scaling circuitry comprises a DAC 1910 coupled to the processor 1906 to convert the signal comprising the digitized wirelessly received ECG unit data to an analogue signal. It may be that the voltage scaling circuitry also comprises, or is coupled to, the reference voltage source 1912 coupled to an input of the DAC 1910 where the DAC 1910 is configured to receive the reference voltage. For example, the reference voltage may be, or may be approximately, 2.5 Volts.

The output of the DAC 1910 may be at least one of attenuated and re-biased for example, by at least one of attenuator 1918 (which may, for example be a −34 dB attenuator) and (summing) buffer amplifier 1920, to rescale the output voltage signal of the DAC to be centred around a given reference voltage. The reference voltage around which the voltage signal is centred may be (approximately) equal to half of the reference voltage of supply 1912. For example, the reference voltage around which the voltage signal is centred may be (approximately) 1.25 Volts.

It may be that the summing amplifier 1920 receives an input from the buffer 1916 of the reference voltage signal generation circuitry. This input may be used to reduce (via summing) the output of the attenuator 1918 to approximately half of the input voltage.

The voltage scaling circuitry and the reference voltage signal generation circuitry are configured, respectively, to provide the scaled voltage signal and the reference voltage signal to respective first and second connectors, or input lines, of the patient monitor configured to receive the wired connection input signals from the ECG unit.

The re-centred voltage signal may be output to the patient monitor, for example on a first line of the patient monitor. For example, it may be that the voltage signal is a voltage to be applied to a first input line of the patient monitor. It may also be that the voltage scaling circuitry optionally further comprises at least one resistor along the output path 1922a of the output path of the voltage signal to the patient monitor.

The generated reference voltage signal may be output to the patient monitor, for example on a second line of the patient monitor. For example, it may be that the reference voltage signal is a voltage to be applied to a second input line of the patient monitor. It may also be that the reference voltage signal generation circuitry optionally further comprises at least one resistor along the output path 1922b of the output path of the reference voltage signal to the patient monitor.

It may be that the reference voltage is a DC signal, which, for example may be constant in time. It may be that the voltage difference between the first and second input lines of the patient monitor resulting from the voltage signals output by the voltage scaling circuitry and the reference voltage signal generation circuitry emulates, or reproduces, the potential measured between the first and second electrodes of the ECG unit.

It is be understood that the voltage scaling circuitry and the voltage signal generation circuitry shown in FIG. 19 is an example for illustrative purposes the voltage scaling circuitry and the voltage signal generation circuitry is not so limited.

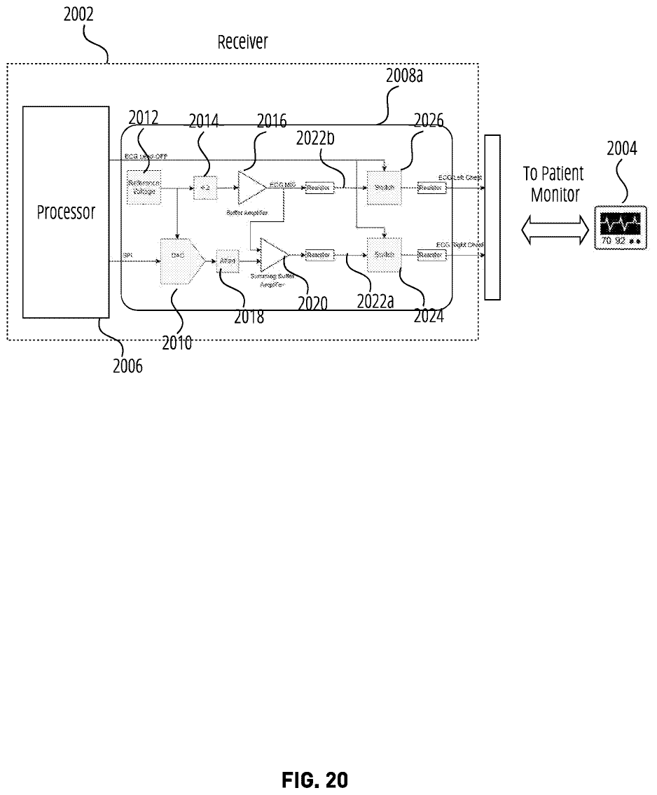

FIG. 20 shows a schematic circuit diagram of an example receiver 2002. The receiver 2002 may comprise all or some of the circuitry of receiver 1902.

In addition to voltage scaling circuitry and the reference voltage signal generation circuitry, the signal generation circuitry 2008a comprises switching circuitry controlled by the processor 2006 to emulate a lead-off event at the patient monitor 2004.

It may be that, as illustrated in FIG. 20, the switching circuitry comprises switch 2024 in the output path 2022a and switch 2026 in the output path 2022b. Switches 2024 and 2026 may be coupled to an ECG lead-off signal to be output by processor 2006. It may be that switch 2024 and switch 2026 are respectively configured, responsive to receiving an ECG lead-off signal indicative that a lead-off event has occurred (e.g., a confirmation of the event) to open the circuit, i.e., to interrupt the output paths 2022a and 2022b. For example, the switches 2024 and 2026 may open to prevent electrical communication along the output paths 2022a,b to the patient monitor. This may emulate a lead-off event at the ECG unit, or between the ECG unit and at least a respective one of the electrodes thereof.

It may be that the processor 2006 is configured, based on detecting a lead-off event characteristic in the received ECG data to generate the ECG lead-off signal. For example, the processor 2006 may be configured, in response to positively detecting a lead-off event characteristic in the received ECG data, to generate a lead-off signal that positively indicates (e.g., confirms) a lead-off signal, or otherwise causes the switching circuitry to create an open circuit. Alternatively, for example, the processor 2006 may be configured, in response to failing to detect a lead-off event characteristic in the received ECG data, to generate a lead-off signal that negatively, or does not positively indicate a lead-off signal, or otherwise causes the switching circuitry to maintain electrical communication along the output paths 2022a,b.

It may be that the lead-off event characteristic in the ECG data is an impedance exceeding a pre-determined threshold. However, the lead-off event characteristic in the ECG data is not so limited and may comprise any parameter (or value thereof) that indicates a lead-off event.

Alternatively, it may be that the lead-off event is detected by a wireless transmitter connected to the ECG unit, by way of which the ECG data is wirelessly received. For example, the transmitter may transmit the ECG lead-off signal to the receiver 2002 and, thereby, the processor 2006. The wireless transmitter may detect the lead-off event as described above, e.g., based on a lead-off event characteristic, such as an impedance exceeding a threshold.

It is be understood that the switching circuitry shown in FIG. 20 is an example for illustrative purposes the switching circuitry is not so limited.

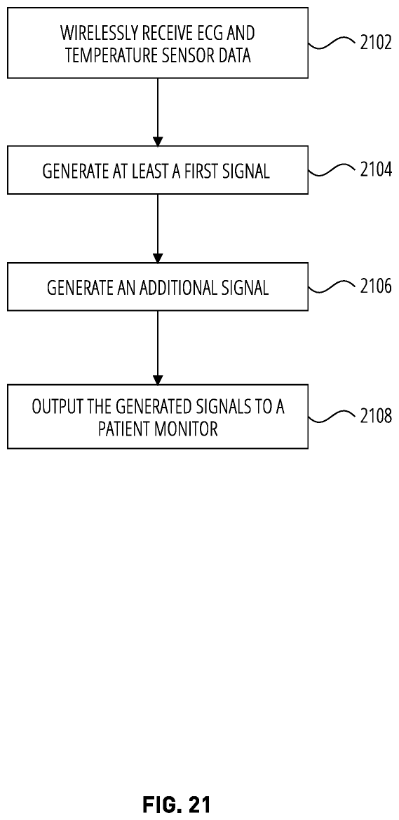

FIG. 21 is a flow chart illustrating an example method to be performed by a receiver connectable to a patient monitor. For example, any of the receivers illustrated in FIGS. 15 to 20.

In block 2102 the method comprises wirelessly receiving electro-cardiogram, ECG, data sensed by an ECG unit and temperature sensor data sensed by a temperature sensor unit.

In block 2104 the method comprises generating at least a first signal based on the received ECG data. The at least first signal is configured to emulate wired connection input signals received by the patient monitor from the ECG unit.

In block 2106 the method comprises generating an additional signal based on the temperature sensor data. The additional signal is configured to emulate a wired connection input signal received by the patient monitor from the temperature sensor unit.

In block 2108 the method comprises outputting the generated signals to the patient monitor.

The ECG and temperature sensor data may be as described above. The generated signals, the at least first signal, and the additional signal may also be as described above.

It may be that wirelessly receiving the ECG data and the temperature sensor data comprises wirelessly receiving at least digitized and packetized ECG and temperature sensor data in respective packets. It may be that each packet has a corresponding header.

It may be that the method comprises wirelessly receiving the data via any wireless communication standard. For example, receiving the data from the temperature sensor unit and/or the ECG unit by way of a wireless transmitter connected thereto via any wireless communication standard for operation in a short-range communication network, such as a wireless local access network (WLAN), using e.g., Wi-Fi® (IEEE 802.11), or for any wireless communication standard for operation with via a Wireless Personal Area Network (WPAN) or low-power personal area network (LPPAN), using, for example, any IEEE 802.15 wireless standard including Bluetooth® (IEEE 802.15.1), or Zigbee® (IEEE 802.15,4), or any modifications or updates thereto. However, the method is not so limited and may alternatively comprise receiving the data wirelessly using other short-range communication networks, such as e.g., infrared data association (IrDA).

It may be that generating the additional signal comprises generating a signal, based on the wirelessly received (analogue) voltage signal (that is based on, or indicates, a resistance controlled by the temperature sensor) to draw a current from the patient monitor that causes a signal generation circuit pathway of the receiver (e.g., the pathway between the processor and the patient monitor input configured to receive the wired connection from the temperature sensor unit, or a portion thereof) to have an impedance that emulates, or reproduces, the resistance of the temperature sensor of the temperature sensor unit.

For example, it may be that the generating the additional signal comprises sensing a current in the signal generation circuit pathway between the processor of the receiver and a connector of the patient monitor configured to receive the wired connection input signal from the temperature sensor unit, and providing the sensed current to the processor. The method may further comprise sensing a voltage across the signal generation circuit pathway between the processor and the connector of the patient monitor and providing the sensed voltage to the processor. The method may further comprise calculating, by the processor, based on the sensed voltage and the temperature sensor data, a current to be drawn from the patient monitor, wherein the current to be drawn causes the signal generation circuit pathway to have an impedance that emulates the resistance of a temperature sensor of the temperature sensor unit. The method may comprise adjusting, based on the sensed current, the current drawn from the connector of the patient monitor to draw (e.g., to match) the calculated current.

It may also be that generating the additional signal comprises providing a feedback signal (e.g., a feedback voltage signal) to the processor. For example, to indicate the voltage at which the patient monitor drives the temperature sensor (or would drive the temperature sensor if wired to the temperature sensor unit). The method may also comprise, based on the voltage at which the patient monitor drives (or would drive) the temperature sensor unit, dynamically adapting the current drawn by the signal generation circuitry, for example to account for, and e.g., emulate, variations in the voltage at which the patient monitor drives the temperature sensor unit. It may be that this feedback process improves the retrofitting of the receiver to the patient monitor and/or the temperature sensor unit, as it may allow the receiver to be adaptable to different types of patient monitors and/or temperature sensor units, or to different operations of patient monitor and/or temperature sensor units. Additionally, or alternatively, it may be that a feedback current signal is provided to the processor to enable the processor to account for changes between an intended current to be drawn from the patient monitor (as calculated by the processor), and an actual current drawn from the patient monitor (as measured from the circuitry).

It may be that generating at least a first signal based on the received ECG data comprises scaling a voltage signal based on the received ECG data such that the scaled voltage signal is centred around a given reference voltage. Generating at least a first signal based on the received ECG data may further comprise generating a constant reference voltage signal at the given reference voltage and providing the scaled voltage signal and the reference voltage signal to respective first and second connectors of the patient monitor configured to receive the wired connection input signals from the ECG unit.

It may be that the method further comprises controlling by the processor of the receiver switching circuitry to emulate a lead-off event at the patient monitor. For example, controlling by the processor the switching-circuitry to emulate a lead-off event in response to detecting a lead-off characteristic in the ECG data, such as detecting that an impedance is above a predetermined threshold.

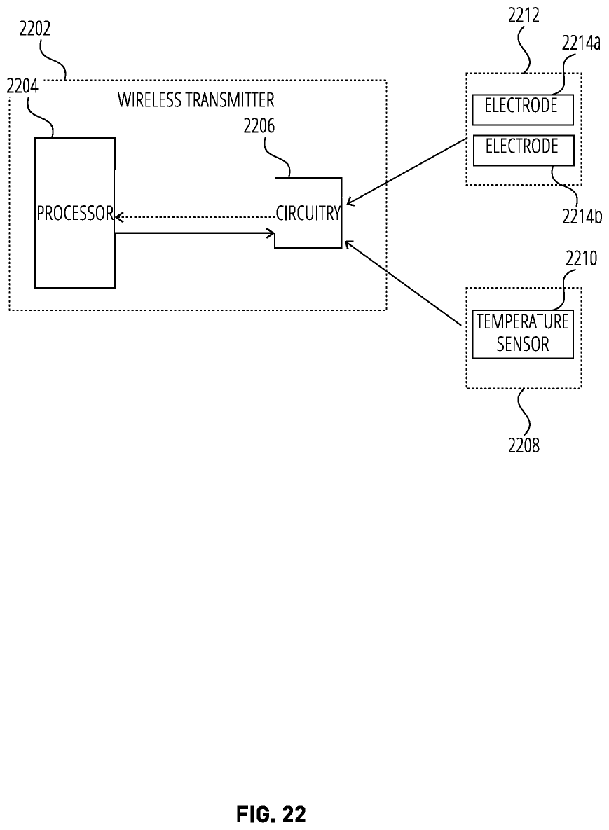

FIG. 22 shows a schematic diagram of functional blocks of an example transmitter connectable to a temperature sensor unit and an ECG unit.

As described above, the temperature sensor unit 2208 comprises at least a temperature sensor 2210 and the ECG unit 2212 comprises at least two electrodes 2214a,b.

The circuitry 2206 is configured to receive ECG data from the ECG unit and receive temperature sensor data from the temperature sensor unit. The circuitry 2206 and processor 2204 are configured to generate a digitized and packetized signal comprising the ECG and temperature sensor and wirelessly transmit the signal. For example, wirelessly transmit the signal to one of the receivers described herein with respect to FIGS. 15 to 20.

It may be that the signal comprises ECG data and temperature sensor data packetized in respective packets, wherein each packet has a corresponding header. For example, it may be that the processor 2204 and/or circuitry 2206 is (are) configured to generate a signal comprising ECG data and temperature sensor data packetized in respective packets, wherein each packet has a corresponding header.

It may be that the transmitter is also configured to wirelessly transmit an ECG lead-off signal. It may also be that the transmitter is configured to detect a lead-off event. For example, the wireless transmitter may detect the LED-off event as described above, e.g., based on a lead-off event characteristic, such as an impedance exceeding a threshold.

It may be that the circuity 2206 is coupled to an antenna (not shown) configured to wirelessly transmit the generated signal.

The transmitter 2202 may be any general purpose transmitter configured to receive data and digitize and packetize data for wireless transmission.

It may be that the transmitter is configured to wirelessly transmit the signal using any wireless radio communication protocol, such as those described herein.

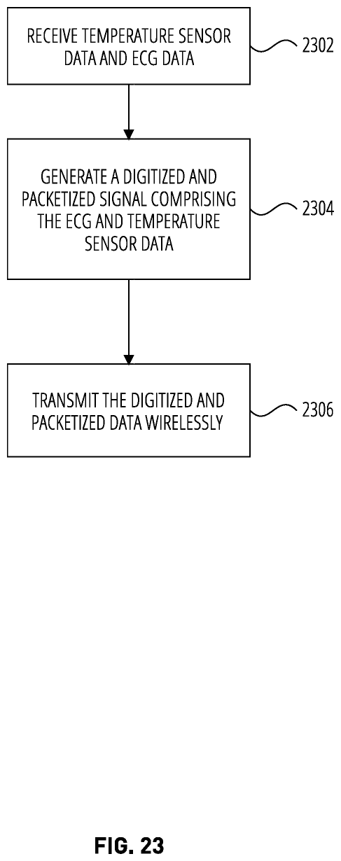

FIG. 23 is a flow chart illustrating an example method to be performed by a transmitter connectable to a temperature sensor unit and an ECG unit.

In block 2302 the method comprises receiving the temperature sensor data from the temperature sensor unit and receiving the ECG data from the ECG unit.

It may be that the transmitter receives the data from the temperature sensor 2210 or unit and/or the ECG unit over a wired connection. For example, the transmitter may receive at least one voltage signal (i.e., voltage values as a function of time) over respective wired connections from the ECG unit and/or the temperature sensor unit.

In block 2304 the method comprises digitizing and packetizing the received data (e.g., the received voltage values) for wireless transmission. It may be that the digitization and packetization of the received temperature sensor data and ECG data may be performed by any well-known radio signal processing method and means, and may for example conform to any short-range wireless communication standard protocol requirements. It may be that the temperature sensor data and ECG data are packetized in respective packets. For example, in consecutive packets. It may be that the method comprises generating, and inserting into the signal, header information indicating the data (e.g., type of data, temperature sensor or ECG data) in the respective packets.

In block 2306 the method comprises wirelessly transmitting the digitized and packetized signal (e.g., to the receiver). The method may comprise wirelessly transmitting the signal via an antenna. The method may comprise wirelessly transmitting the signal using any short-range wireless communication standard protocols.

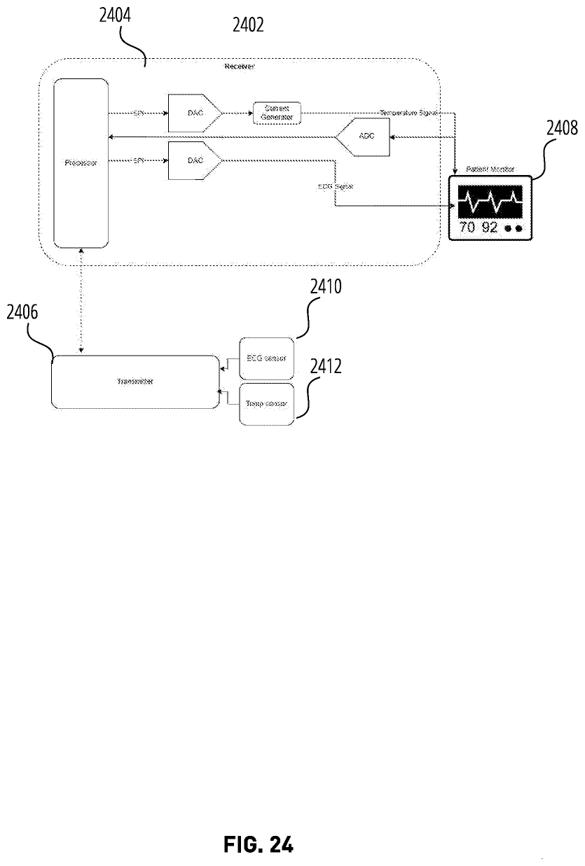

FIG. 24 shows a schematic diagram of functional blocks of an example system 2402. The system 2402 comprises a receiver 2404 connectable to a patient monitor 2408 that may be any of the example receivers described above, for example with respect to FIGS. 15 to 20.

The system 2402 further comprises a transmitter 2406 connectable to an ECG unit 2410 and a temperature sensor unit 2412. The transmitter may be any of the transmitters described above, for example with respect to FIG. 22.

The system 2402 may further comprise the patient monitor 2408. Alternatively, or additionally, the system may further comprise the ECG unit 2410 and/or temperature sensor 2412, or components thereof. For example, the system 2402 may alternatively, or additionally, comprise at least two electrodes, and/or a temperature sensor (e.g., a thermistor).

The system 2402 may alternatively, or additionally comprise at least a pulse oximetry unit (not shown), or first and second LEDs and photodiode thereof.

The system may alternatively, or additionally comprise a transmitter configured to wirelessly transmit the pulse oximeter data, such as the transmitter described above with respect to FIG. 12.

The system may comprise an additional receiver, such as the receiver described above with respect to FIG. 1 or FIGS. 4 to 10. Alternatively, the receiver 2404 may be further configured to perform the function(s) or may comprise the circuitry described above with respect to any of FIGS. 1 and 4 to 10. For example, a single receiver may be configured to wirelessly receive the pulse oximeter data, the temperatures sensor data and the ECG data and be configured to generate and output respective signals to emulate the effects on the patient monitor of wired connections between the patient monitor and each of the pulse oximeter unit, temperature sensor unit and ECG unit.

It may be that in addition to sensing changes in a potential difference across the at least two electrodes of the ECG unit caused by the electrical activity of a patient's heart, the ECG unit may also sense, by way of the electrodes, an (electrical) impedance (e.g., a time varying impedance) across the patient's chest wall caused by respiratory actions. For example, it may be that the movement in a patient's chest wall during respiration may result in measurable changes in the impedance sensed by the electrodes attached on opposing sides of the patient's body (i.e., with the patient's chest wall therebetween). It may be that the changes in impedance (or impedance signal) are cyclical due to the underlying respiration cycle. It may be that the impedance signal can be described by a waveform. It may be that the impedance signal resulting from respiratory actions modulates the ECG data indicating the electrical activity of the patient's heart. For example, the ECG data received by the example receivers described herein may comprise indications of both the electrical activity of the patient's heart, and the respiratory actions of the patient.

It may be that the time-varying impedance (or impedance signal) indicates at least one characteristic of respiration, such as a respiration rate of the patient. In this way, the impedance signal may indicate a vital sign of the patient. For example, at least one of the amplitude and morphology of the impedance signal waveform may provide clinical information, such as indications of health, or deviations therefrom.

It may be that the ECG unit is configured to measure the impedance (signal) between the at least two electrodes. It may also be that the ECG unit, or the wireless transmitter connected thereto, is configured to determine at least one parameter value characterising a waveform representing the impedance signal. For example, the ECG unit, or wireless transmitter, may be configured to determine an I value (cosine component of the waveform) and a Q value (sine component of the waveform), or any other parameter values that describes or characterises a waveform.

For example, it may be in order to detect the electrical signals indicative of the electrical activity of the patient's heart, a potential difference is measured across the at least two electrodes of the ECG unit. The electrical activity of the patient's heart may act on, or change the potential difference between the at least two electrodes. This change may be measured by the at least two electrodes of the ECG unit and the change may indicate the electrical activity of the patient's heart.

It may also be that a current signal is applied to the first and second electrodes of the ECG unit for indicating a respiratory rate of the patient. For example, the current signal may be applied to first and second electrodes on opposing sides of the patient's heart. It may be that the current signal is characterised by a waveform with a high frequency. For example, the current signal may have a frequency between 20 to 100 kHz, and may be typically 50 kHz. It may be that a voltage measured between the electrodes may be used to determine an impedance between the first and second electrodes, which may be indicative of a patient chest wall impedance. It may be that the impedance is measured as a function of time to determine an impedance signal that may indicate a respiratory rate of a patient.

It may be that the high frequency current signal is, or is considered to be an impedance excitation signal. It may be that the signal is continuously applied to the electrodes of the ECG unit, and may be filtered out to detect the changes to the measured voltage across the at least two electrodes resulting from the respiratory actions as opposed to resulting from the electrical activity of the patient's heart, as is known from conventional ECG unit operation. Alternatively, it may be that the signal is applied to the electrodes of the ECG unit at intervals, such that the impedance measurements are inter-sampled with the ECG measurements. It may be that the at least one parameter value determined by the ECG unit, or wireless transmitter connected thereto, characterises the impedance signal waveform determined from the high frequency current signal (i.e., the impedance excitation signal). For example, the transmitter 2202 described above, may be further configured to determine and transmit the at least one parameter value.

The at least one parameter value may be transmitted for reception by a receiver, such as any of receivers described above with respect to FIGS. 15 to 20, which are configured to receive ECG data.

It may be that any of the receivers described above with respect to FIGS. 15 to 20, which are configured to receive ECG data are also configured to receive at least one parameter value that describes an impedance signal waveform indicative of a respiratory measurement (e.g., measured by way of the electrodes of the ECG unit). For example, the receivers may be configured to receive I and Q values describing the impedance signal waveform.

It may be that any of the receivers described above with respect to FIGS. 15 to 20, which are configured to receive ECG data are also configured to control, or to modulate the at least one ECG signal generated and output to the patient monitor (i.e., the at least first generated signal configured to emulate at least a first wired connection input signal received by the patient monitor from the ECG unit that indicates the electrical activity of the patient's heart) to emulate, or reproduce, the impedance signal of the patient (i.e., the impedance of the chest wall of the patient, that varies with time). For example, the receivers described above with respect to FIGS. 15 to 20, may be configured to control, or to modulate the at least one ECG signal generated and output to the patient monitor (indicative of the electrical activity of the patient's heart), to emulate, or reproduce, the impedance signal across the first and second input lines of the patient monitor that are configured to be wired to the ECG unit, to reproduce the effect at the patient monitor of the input lines being wired to the ECG unit.

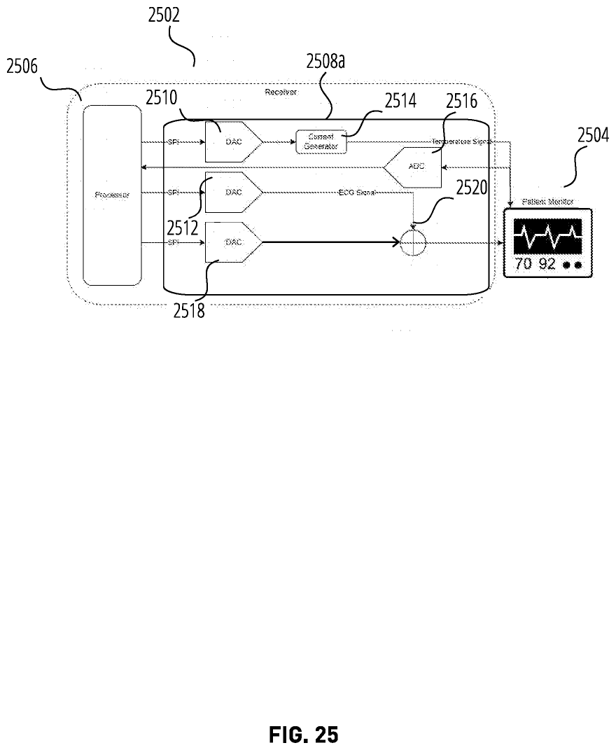

FIG. 25 shows a schematic circuit diagram of an example receiver 2502. The receiver 2502 may comprise the same functional blocks as receiver 1502, receiver 1602 and may comprise some or all of the circuitry of receiver 1702.

The receiver 2502 is configured to receive at least one parameter value that describes an impedance signal waveform indicative of a respiratory measurement (e.g., measured by way of the electrodes of the ECG unit). For example, the receiver 2502 is configured to receive I and Q values describing the impedance signal waveform.

The receiver 2502 is configured to control, or to modulate the at least one ECG signal generated and output to the patient monitor by any one of receivers 1502, 1602, 1702, 1902, 2002, (i.e., the at least first generated signal configured to emulate at least a first wired connection input signal received by the patient monitor from the ECG unit that indicates the electrical activity of the patient's heart) to emulate, or reproduce, the impedance signal of the patient (i.e., the impedance of the chest wall of the patient, that varies with time). For example, the receiver 2502 is configured to control, or to modulate the at least one ECG signal generated and output to the patient monitor (indicative of the electrical activity of the patient's heart), to emulate, or reproduce, the impedance signal across the first and second input lines of the patient monitor that are configured to be wired to the ECG unit, to reproduce the effect at the patient monitor of the input lines being wired to the ECG unit.

The receiver 2502 comprises signal generation circuitry 2508a (such as at least signal generation circuitry 1608a, 1708a, 1908a or 2008a) that is configured to control (or modulate) an impedance signal (for example, by introducing impedance) of a signal generation pathway (e.g., between the processor and the first and second input lines of the patient monitor 2504, or respective output lines, or output paths, of the receiver 2502 connectable to the input lines of the patient monitor 2504). For example, the signal generation circuitry may control the impedance based on the received at least one parameter value.

The receiver 2502 is configured to control or modulate an impedance (signal) in a signal generation pathway (e.g., between the first and second input lines to the patient monitor 2504, or respective output lines of the receiver 2502 connectable to the input lines of the patient monitor 2504) to emulate the impedance (corresponding to the impedance of the patient chest wall) between the first and second input lines of the patient monitor in a wired connection configuration.

The processor 2506 is configured to drive the signal generation circuitry based on the received at least one (digitized) parameter value. For example, the processor 2506 may drive the signal generation circuitry to control (or modulate) the impedance of a signal generation pathway (e.g., between the processor and the first and second input lines of the patient monitor 2504, or corresponding output lines/paths of the receiver 2502) based on the received at least one parameter value.

For example, it may be that processor 2506 is configured to drive the signal generation circuitry to control (or modulate) the impedance of the signal generation pathway (e.g., between the processor and the first and second input lines of the patient monitor or corresponding output lines of the receiver) based on the received at least one parameter value to emulate or reproduce the impedance signal between the first and second input lines of the patient monitor when the ECG unit is wired to the patient monitor, and therefore to emulate the time-varying impedance across the patient's chest wall.

The signal generation circuitry 2508a comprises a DAC 2518 to convert the received at least one parameter value (e.g., I and Q values) to an analogue voltage signal, for example a voltage signal representative of the impedance across the patient's chest wall sensed by the ECG unit. For example, the processor 2506 may generate a voltage signal (indicative of the impedance signal waveform sensed by the ECG unit) based on the received at least one parameter value. For example, the voltage signal may indicate, or emulate, at least one of the morphology, and amplitude of the impedance signal waveform, or any impedance signal waveform characteristic that may provide vital sign (or clinical) information.

The signal generation circuitry 2508a comprises circuitry 2520 to modulate the at least one generated ECG signal (to be output to the first and second input lines of the patient monitor) to control the impedance of a pathway of the signal generation circuitry 2508a (e.g., between the first and second input lines of the patient monitor or corresponding output lines of the receiver) to emulate the impedance signal (or impedance signal waveform) sensed by the ECG unit. For example, the circuitry 2520 of the signal generation circuitry 2508a may modulate the generated first and/or second signals to be output to the respective first and second input lines of the patient monitor.

It may be that the circuitry 2520 to control the impedance of a pathway of the signal generation circuitry 2508a is driven, or controlled, by the processor 2506, or output therefrom, or, for example, the output of the DAC 2518 coupled to the processor 2506. It may be that the circuitry 2520 is signal addition circuitry.

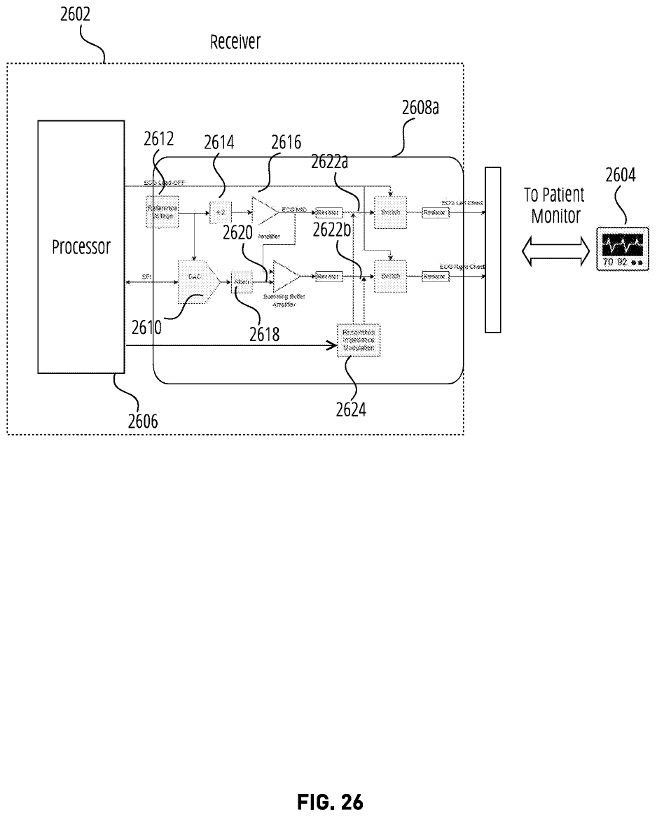

FIG. 26 shows a schematic circuit diagram of an example receiver 2602. The receiver 2602 may comprise all or some of the circuitry of receiver 2502, receiver 1902 or receiver 2002.

Receiver 2602 comprises circuitry 2624 (e.g., impedance controlling or modulating circuitry) configured to modulate the generated at least first signal configured to emulate at least a first wired connection input signal received by the patient monitor from the ECG unit, or, for example, first and second signals to be output to the first and second input lines of the patient monitor. The circuitry 2624 is coupled to the signal generation circuitry at output path 2622b and 2622b. As illustrated in FIG. 26, the circuitry 2624 is coupled to the respective output paths 2622b and 2622b at a point at which the input signals to the circuitry 2624 correspond to the emulated signals to be provided to the input lines of the patient monitor (e.g., the signals are reference voltage and scaled voltage signals), i.e., along a point in the output paths after the buffer amplifiers 2616 and 2618. The circuitry 2624 may be coupled to the respective output paths 2622b and 2622b before, or after any optional resistors along the output paths. The circuitry 2624 may be coupled to the respective output paths 2622b and 2622b before, or after the optional switches configured to emulate a lead-off event.

As described above, it may be that the circuitry 2624 to control the impedance of a pathway of the signal generation circuitry 2608a is driven, or controlled, by the processor 2606, or output therefrom, or, for example, the output of a DAC coupled to the processor 2606.

It may be that the circuitry 2624 comprises at least one resistive element controlled by the processor 2606, or output therefrom, or, for example, an output of an DAC coupled to the processor 2606 to control the impedance in a pathway of the signal generation circuitry 2608a to emulate an impedance signal between the first and second input lines of the patient monitor resulting from a wired connection between the patient monitor and the ECG unit.

For example, the at least one resistive element of circuitry 2624 may be controlled based on the impedance signal waveform detected by the ECG unit and indicated (or represented) by the voltage signal generated by the processor 2606 (based on the received at least one parameter value) to control the impedance in the pathway of the signal generation circuitry 2608a.

It may be that the at least one resistive element is controlled by the output from the processor 2606 (i.e., voltage signal). For example, the voltage signal output by the processor 2606 may vary the resistance in the path of the signal generation circuitry 2608a with time to emulate the impedance signal sensed by the ECG unit.

It may be that the at least one resistive element comprises a FET, e.g., a MOSFET, wherein the voltage signal based on the received at least one parameter value is provided to the gate of the MOSFET to control the impedance of the signal generation circuitry pathway. For example, the MOSFET may be controlled by applying the voltage signal to the gate of the MOSFET. It may be that the circuitry 2624, is configured to provide an impedance signal or waveform thereof with a baseline (i.e., DC offset) with an impedance within a range within which the patient monitor may be configured to operate. For example, the circuitry 2624 may be configured to provide an impedance signal with a baseline between 100 and 5,000 Ohms.

More particularly, it may be that the circuitry 2624 is configured provide an impedance signal or waveform thereof with a baseline (i.e., DC offset) at, or around the typical chest wall impedance of a patient, for example, approximately 560 Ohms. It may be that the MOSFET is controlled, based on the voltage signal (which is based on the received at least one parameter value, and is representative of the impedance signal waveform measured by the ECG unit) to vary the amplitude of the impedance signal provided to the signal generation circuitry pathway. For example, the amplitude of the impedance signal waveform generated by way of the MOSFET may be varied in response to the voltage signal applied to the gate to control the MOSFET channel. For example, the amplitude of the generated impedance signal waveform may be increased as the voltage applied to the gate approaches and reaches the drain voltage of the MOSFET, and may be reduced as the voltage signal falls below the drain voltage of the MOSFET. It may be that the drain voltage of the MOSFET is biased to approximately 2.5 Volts.

It may be that the at least one resistive element comprises two back-to-back MOSFETS with a shared, or common, gate. It may be that the resistance, and thereby the generated impedance signal or waveform thereof is controlled by the voltage signal (output by the processor or DAC coupled to the processor) provided to the common gate.

It may be that in addition to an amplitude of the impedance signal waveform, other characteristics of the waveform may provide clinical information, such as for example, the morphology of the waveform (e.g., the shape or slope of the rising and falling edges etc.). It may be that due to the non-linear nature of a FET, the use of a FET as the resistive element, or component thereof, requires additional signal processing of the generated impedance signal to emulate the characteristics (e.g., morphology) of the impedance signal waveform sensed by the ECG unit from the patient. It may be that such signal processing prevents signal degradation, or deviations resulting from the signal re-constitution, from being interpreted as clinical indications of deviations from health etc. For example, the circuitry 2624 may optionally further comprise signal processing circuitry. The signal processing circuitry may include at least one of buffering circuitry, filtering circuitry or processing circuitry, such as firmware. The processing circuitry may be configured to e.g., smooth and/or interpolate, or otherwise improve the emulation of the impedance signal waveform sensed from the patient.

It may be that the at least impedance controlling circuitry or the at least one resistive element thereof, is coupled to the first and/or second output paths 2622b, 2622a of the signal generation circuitry couplable to the first and second input lines of the patient monitor configured to receive the wired connections from the ECG unit.

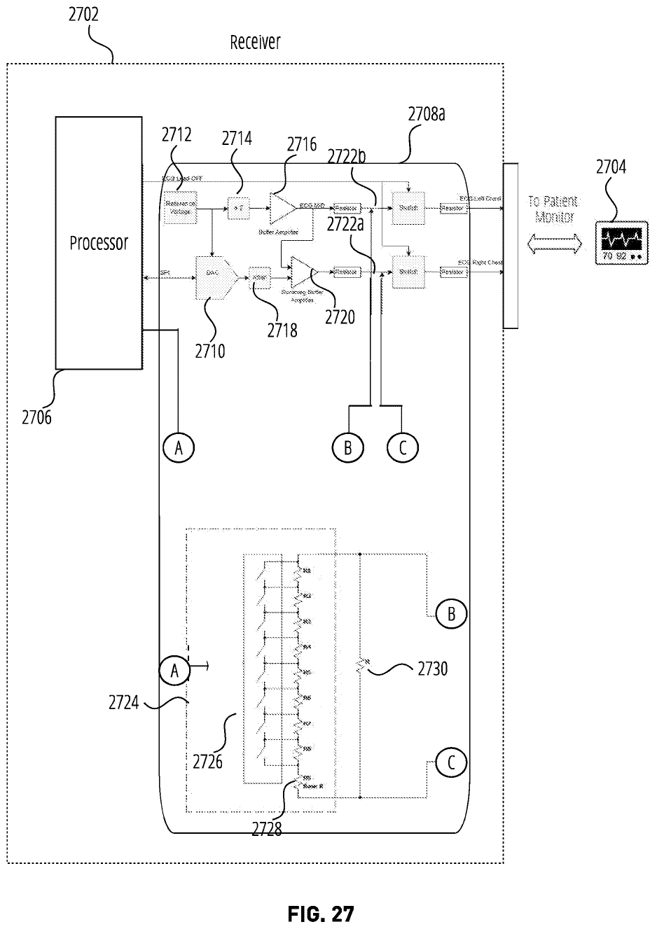

FIG. 27 shows a schematic circuit diagram of an example receiver 2702. The receiver 2702 may comprise all or some of the circuitry of receiver 2602.

The signal detection circuitry 2708a comprises a resistor network 2724. The resistor network 2724 may form part of the impedance controlling circuitry of receiver 2702, and may be, or form part of, the at least one resistive element of receiver 2702. The resistor network 2724 is controlled by the output from the processor 2706 to emulate an impedance signal (or waveform thereof) at the patient monitor. The input of the resistor network 2724 is coupled to an output from the processor 2706. The respective outputs of the resistor network 2724 are coupled to output paths 2722a and 2722b of the signal generation circuitry.

In order to aid illustration, in FIG. 27, the input of the resistor network 2724 is shown as coupled to the output of the processor 2706 by way of connection node A, and the outputs of the resistor network 2724 are shown as coupled to the output paths of the signal generation circuitry 2722b and 2722a by connector nodes B and C, respectively. However, the receiver 2702 is not so limited and the resistor network 2724 may connected to the processor 2706 and output paths 2722a, 2722b via any configuration.

The resistor network 2724 comprises a plurality of resistors 2726 connected in series. The plurality of resistors is operable (i.e., controllable by) a plurality of switches. For example, each of the plurality of resistors may be controlled by a respective switch. It may be that the plurality of switches are controlled by the processor 2706 (or output therefrom). The processor 2706 may be configured (for example via firmware), based on the received at least one parameter value, to control the respective switches of the plurality of resistors to control the impedance of the signal generation circuitry pathway to emulate the impedance signal waveform at the patient monitor.

The resistor network 2724 illustrated in FIG. 27 comprises eight resistors (and corresponding switches) connected in series. However, the resistor network is not so limited and may comprise fewer, or more resistors connected in series. For example, the resistor network may comprise six, seven, nine, ten, or more resistors connected in series.

The resistor network 2724 illustrated in FIG. 27 is further coupled in series to an additional resistor, e.g., a base resistor 2728.

It may be that the number of the plurality of resistors 2726 connected in series controls the resolution of the emulated impedance signal waveform output to the patient monitor, i.e., the size of the impedance increments that can be distinguished (the difference between consecutive impedance values, AOhms, that can be distinguished, and subsequently indicated by the patient monitor). For example, the number of distinguishable impedance values within a given range may be equal to, where n is the number of the plurality of resistors 2726 connected in series (not including the base resistor 2728). It may be that eight resistors connected in series provides sufficient resolution such that the patient monitor indicates an impedance signal waveform that can be analysed by a medical professional to form a clinical picture, or sufficiently indicates a vital sign of the patient. It may be that the resolution provided by less than eight resistors is sub-optimal for indicating a vital sign.

It may be that the values selected for (each of) the plurality of resistors 2726 connected in series defines the range of impedance values that can be generated by the plurality of resistors 2726 connected in series.

It may be that the base resistor 2728 modifies the range of impedance values output by the plurality of resistors of the resistor network connected in series. For example, the base resistor 2728 may add a linear resistance value to the range generated by the plurality of resistors connected in series.

Optionally, it may be that the resistor network 2724 is connected in parallel to at least one resistor 2730. It may be that connecting the resistor network 2724 in parallel to a resistor allows further modifying (for example re-scaling) of the range of impedance values output by the impedance controlling circuitry.

It may be that the impedance range output by the resistor network (and optionally the parallel resistor) defines the peak-to-peak amplitude of the impedance signal waveform, i.e., the difference in impedance (Ohms) between the peak and trough of the impedance signal waveform amplitude.

It may be that the parallel resistor value is set at, or around the typical impedance of the chest wall of a patient, for example at, or around 560 Ohms. This may set the baseline of the impedance signal waveform. It may be that the impedance range output by the resistor network (and optionally the parallel resistor), and thus the range in peak-to-peak amplitude of the generated impedance signal waveform may be approximately 2 Ohms, of for example between 1.5 and 2.5 Ohms.

It may be that coupling the optional resistor 2730 in parallel to the resistor network 2724 enables the resistor network of the impedance controlling circuitry to be applied to a wider range of systems without requiring re-selection of the values of (each of) the plurality of resistors 2726 in the resistor network. In this way, the retrofitting of the receiver 2702 to a patient monitor and/or an ECG unit may be improved.

It may also be that the impedance signal waveform generated via the resistor network 2724 (to be output to the patient monitor) has a morphology that better (i.e., more accurately) emulates the waveform of the impedance signal sensed by the ECG unit from the patient, for example in comparison to the waveform generated by a resistive element comprising a FET.

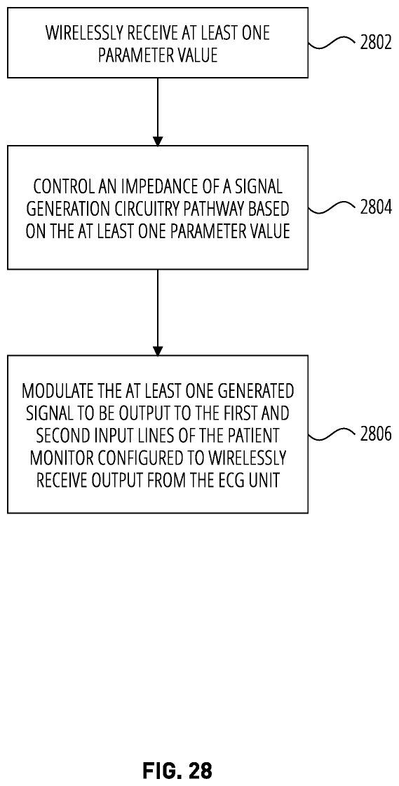

FIG. 28 is a flow chart illustrating an example method to be performed by a receiver connectable to a patient monitor. For example, the receiver illustrated in FIG. 26 or FIG. 27.

In block 2802 the method comprises wirelessly receiving, for an ECG unit, at least one parameter value that describes an impedance signal waveform indicative of a respiratory measurement.

In block 2804 the method comprises controlling an impedance of a signal generation circuitry pathway based on the received at least one parameter value. For example, the impedance of the signal generation pathway may be controlled by the processor based on the received at least one parameter. It may be that impedance is controlled by impedance controlling circuitry coupled to the processor, or a DAC coupled therebetween. It may be that the impedance is controlled by controlling a resistive element of the impedance controlling circuitry. For example, by providing the voltage signal output by the processor, or DAC coupled thereto, to a gate of a FET. Alternatively, the impedance may be controlled by controlling a resistor network by the processor (for example by firmware of the processor), based on the received at least one parameter value.

In block 2806 the method comprises modulating the at least one generated signal to be output to the first and second input lines of the patient monitor configured to wirelessly receive output from the ECG unit. For example, modulating the first and/or second generated signals output to the first and second input lines of the patient monitor configured to wirelessly receive output from the ECG unit.

It may be that the at least one generated signal for output to the input lines of the patient monitor configured to receive the output from the ECG unit over a wired connection is modulated by connecting the impedance controlling circuitry or the at least one resistive element thereof, to the first and/or second output paths of the signal generation circuitry couplable to the first and second input lines of the patient monitor configured to receive the wired connections from the ECG unit.

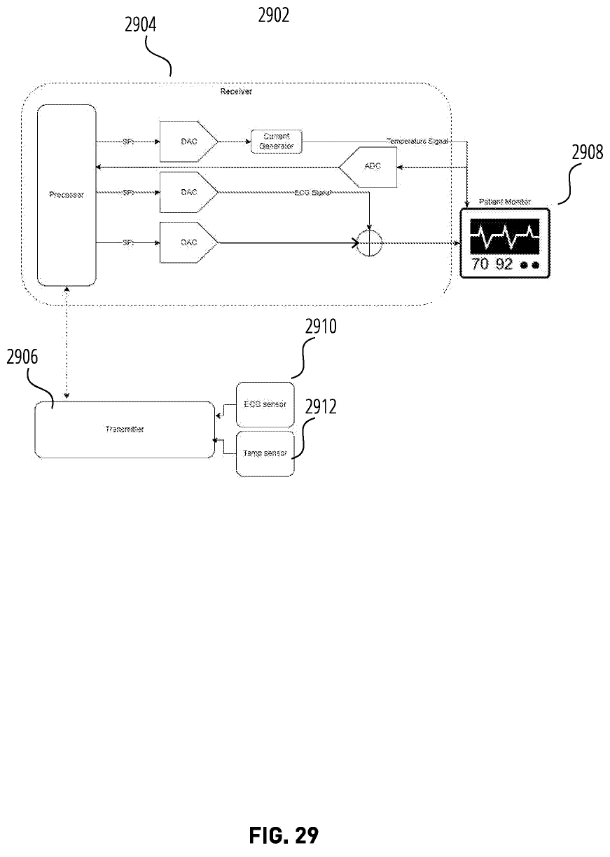

FIG. 29 shows a schematic diagram of functional blocks of an example system 2902. The system 2902 comprises a receiver 2904 connectable to a patient monitor 2908. The receiver may be any one of receivers 2502, 2602, or 2702. The system 2902 further comprises a transmitter 2906 connectable to an ECG unit 2910 and a temperature sensor unit 2912. The transmitter may be the transmitter described above with respect to FIG. 22. The transmitter 2906 may also be configured to transmit at least one parameter value describing an impedance signal waveform detected by the ECG unit.

The system 2902 may further comprise the patient monitor 2908. Alternatively, or additionally, the system may further comprise the ECG unit 2910 and/or temperature sensor unit 2912, or components thereof. For example, the system 2902 may alternatively, or additionally, comprise at least two electrodes, and/or a temperature sensor (e.g., a thermistor).

The system 2902 may alternatively, or additionally comprise at least a pulse oximetry unit (not shown), or first and second LEDs and photodiode thereof.

The system may alternatively, or additionally comprise a transmitter configured to wirelessly transmit the pulse oximeter data, such as the transmitter described above with respect to FIG. 12.

The system may comprise an additional receiver, such as the receiver described above with respect to FIG. 1 or FIGS. 4 to 10. Alternatively, the receiver 2904 may be further configured to perform the function(s) or may comprise the circuitry described above with respect to any of FIGS. 1 and 4 to 10. For example, a single receiver may be configured to wirelessly receive the pulse oximeter data, the temperature sensor data, the ECG data and the respiratory data (e.g., parameter values describing the impedance signal waveform) and may be configured to generate and output respective signals to emulate the effects on the patient monitor of wired connections between the patient monitor and each of the pulse oximeter unit, temperature sensor unit, and the ECG unit (including the ECG signals and the respiratory impedance signal waveform).

The processors described herein, (such as any of processors 406 to 1006, 1204, 1506 to 2006, 2204, or 2506 to 2706) may be a general purpose processor, for example, with a single or with multiple central processing units (CPUs). The processors described herein may be operatively coupled to memory, which may be configured to store instructions, such a machine readable instructions. It may be that the machine readable instructions, when executed by the processor(s) described herein, cause the (respective) processor(s) to perform the method of any of the examples described herein. The machine readable instructions may be provided on a transitory medium such as a transmission medium or on a non-transitory medium such as a storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

In this application, the phrase “at least one of A or B” and the phrase “at least one of A and B” should be interpreted to mean any one or more of the plurality of listed items A, B, etc., taken jointly and severally in any and all permutations.

Where functional units are described as separate circuitry, any of the functionality may be integrated or combined into fewer circuitry.

It is not intended that the order in which the methods are described in this application is to be construed as a limitation, and any number of the described method blocks can be combined in any order to implement the methods or an alternate method. Additionally, individual blocks may be deleted from the methods without departing from the scope of the subject matter described herein.

Within the scope of this application it is expressly intended that the various examples and alternatives set out in the preceding paragraphs, in the claims and/or in the description and drawings, and in particular the individual features thereof, may be taken independently or in any combination. That is, all examples and/or features of any example can be combined in any way and/or combination, for example it may be that apparatus features correspond to method features or vice versa, unless such features are incompatible.

In the above description, for purposes of explanation, specific examples and details thereof are set forth in order to better explain the present invention, as claimed. It is to be understood that such detail is solely for that purpose, and that the appended claims are not limited to the disclosed examples, but, on the contrary, are intended to cover modifications and equivalents that are within the scope of the disclosed examples.

It will be apparent to one skilled in the art that the claimed invention may be practiced using different details than the exemplary ones described herein. Other examples may incorporate structural, logical, electrical, method, and other changes. Portions and features of some examples may be included in, or substituted for, those of other examples. In other instances, well-known features are omitted or simplified to clarify the description of the examples.

The applicant reserves the right to change any originally filed claim or file any new claim accordingly, including the right to amend any originally filed claim to depend from and/or incorporate any feature of any other claim although not originally claimed in that manner.

Example 1. A receiver connectable to a patient monitor, the receiver comprising: a processor; and circuitry coupled to the processor, the circuitry configured to: wirelessly receive data based on an output from a pulse oximeter unit, wherein the data comprises: a first data set comprising voltage values corresponding to a photodiode current sensed responsive to a first LED of the pulse oximeter unit, and a second data set comprising voltage values corresponding to a photodiode current sensed responsive to a second LED of the pulse oximeter unit; generate a signal based on the received data, wherein the signal is configured to emulate a wired connection input signal to the patient monitor; and output the generated signal to the patient monitor.

Example 2. The receiver of Example 1, wherein the voltage values of the respective first and second data set are converted (e.g., by a wireless transmitter connected to the pulse oximeter unit via current to voltage circuitry, such as a transimpedance amplifier) from current values output by the photodiode of the pulse oximeter unit responsive to detecting radiation from the respective first and second LEDs of the pulse oximeter unit.

Example 3. The receiver of Example 1, wherein the first data comprises voltage values corresponding to a photodiode current sensed responsive to the first LED (of the pulse oximeter unit) and from which an ambient light signal has been subtracted, and wherein the second data set comprises voltage values corresponding to a photodiode current sensed responsive to the second LED (of the pulse oximeter unit) and from which an ambient light signal has been subtracted.

Example 4. The receiver of Example 1 or Example 3, wherein the voltage values of the respective first and second data set are converted from current values output by the photodiode of the pulse oximeter unit responsive to detecting radiation from the respective first and second LEDs of the pulse oximeter unit and from which the ambient light signal has been subtracted.

Example 5. The receiver of any one of Examples 1-4, wherein the receiver comprises: a radio communication receiver (e.g., an antenna and radio signal processing circuitry circuitry) to wirelessly receive data from the pulse oximeter unit and wherein the processor is coupled to the radio communication receiver; an LED drive signal detector (e.g., a photodiode); a controller coupled to an output of the LED drive signal detector, the controller to receive a first and a second LED drive signal from the LED drive signal detector corresponding to first and second LEDs of the pulse oximeter unit; a digital to analogue converter, DAC, coupled to the processor to receive an input from the processor; at least one switch coupled to an output of the controller and to an output of the DAC; and an optocoupler coupled to an output of the at least one switch; wherein the at least one switch is to (based on control by the controller) selectively supply a first or second analogue voltage signal output by the DAC, corresponding to the respective first and second LEDs of the pulse oximeter unit, to the optocoupler.

Example 6. The receiver of any one of Examples 1 to 5, wherein the receiver is further configured to: generate a load to emulate a load on the patient monitor by the first and second LEDs of the pulse oximeter unit (e.g., when the first and second LEDs of the pulse oximeter unit are connected via a wire to the patient monitor); and apply the load to the patient monitor.

Example 7. The receiver of any one of Examples 1 to 6, wherein the circuitry comprises: radio communication circuitry configured to wirelessly receive a digitized and packetized signal comprising the data from the pulse oximeter unit; and signal generation circuitry configured to generate the signal to be output to the patient monitor, wherein said signal generation circuitry comprises switching circuitry configured to be controlled to selectively provide the signal to the patient monitor based on a respective one of the first and second data sets of voltage values.

Example 8. The receiver of Example 7, wherein the signal generation circuitry comprises optocoupler circuitry comprising a photodiode (e.g., a first photodiode)configured to provide a current to the patient monitor. Optionally, the optocoupler circuitry also comprises a second photodiode configured to provide a current to the processor to indicate a drive level of the optocoupler.

Example 9. The receiver of Example 7 or Example 8, wherein the signal generation circuitry comprises: digital-to-analogue conversion, DAC, circuitry configured to convert the respective received first and second voltage data sets to respective first and second analogue voltage signals; LED signal detection circuitry configured to detect respective first and second LED drive signals from the patient monitor, wherein the first and second LED drive signals correspond to the first and second LEDs of the pulse oximeter unit respectively; control logic; and optocoupler circuitry; wherein the LED signal detection circuitry is configured to provide the first and second LED drive signals to the control logic, wherein the control logic is configured to select which of the first or second analogue voltage signals to provide to the optocoupler circuitry at a given timestep based on the detected first and second LED drive signals, wherein the optocoupler circuitry is driven based on the selected analogue voltage signal and is configured to provide an emulated wired connection photodiode response to the patient monitor.

Example 10. The receiver of Example 9, wherein the first and second LED drive signals are respective first and second LED drive phase signals.

Example 11. The receiver of Example 8 or Example 9, wherein the LED signal detection circuitry is further configured to provide a current feedback signal to the processor.

Example 12. The receiver of any one of Examples 9 to 11, wherein the control logic is further configured to select an analogue voltage signal corresponding to the photodiode response to an ambient light signal to provide at a given timestep as output to the optocoupler circuitry responsive to an indication that neither the first nor second LED of the pulse oximeter unit are being driven by the patient monitor at that timestep. For example, responsive to an indication (e.g., a signal) from the processor, or responsive to detecting by the control logic based on the respective first and second LED drive signals from the patient monitor that neither the first nor second LED unit are being driven.

Example 13. The receiver of any one of Examples 7 to 12, wherein the signal generation circuitry further comprises respective first and second LEDs to emulate the load on the patient monitor by the first and second LEDs of the pulse oximeter unit. For example, first and second LEDs that are the same as, or similar to the respective first and second LEDs of the pulse oximeter unit. For example, first and second LEDs that emit radiation at the same or similar wavelength (range) as the respective first and second LEDs of the pulse oximeter unit.

Example 14. The receiver of Example 13, wherein the first and second LEDs are connectable to first and second output lines of the patient monitor (e.g., the output lines of the patient monitor configured to be wired to the pulse oximeter unit), and a photodiode of optocoupler circuitry of the signal generation circuitry is connectable to a first input line of the patient monitor (e.g., the input line of the patient monitor configured to be wired to the pulse oximeter unit).

Example 15. A method to be performed by a receiver connectable to a patient monitor, the method comprising: wirelessly receiving data based on an output from a pulse oximeter unit, wherein the data comprises: a first data set comprising voltage values corresponding to a photodiode current sensed responsive to a first LED of the pulse oximeter unit; and a second data set comprising voltage values corresponding to a photodiode current sensed responsive to a second LED of the pulse oximeter unit; generating a signal based on the received data wherein the signal is configured to emulate a wired connection input signal to the patient monitor; and outputting the generated signal to the patient monitor.

Example 16. The method of Example 15, further comprising: generating, by the receiver, a load to emulate a load on the patient monitor by the first and second LEDs of the pulse oximeter; and applying the load to the patient monitor.

Example 17. A transmitter connectable to a pulse oximeter unit, the transmitter comprising: a processor; and circuitry, coupled to the processor, the circuitry configured to: receive a photodiode response signal from the pulse oximeter unit; generate a first data set comprising voltage values corresponding to a photodiode current sensed responsive to a first LED of the pulse oximeter unit based on the received photodiode response signal and a first LED drive signal (e.g., by way of buffering, and optionally by way of current to voltage conversion); generate a second data set comprising voltage values corresponding to a photodiode current sensed responsive to a second LED of the pulse oximeter unit based on the received photodiode response signal and a second LED drive signal (e.g., by way of buffering); generate a digitized and packetized signal comprising the first and second data sets; and wirelessly transmit the signal (e.g., using radio communication protocols).

Example 18. The transmitter of Example 17 comprising: a processor to receive the photodiode response signal and the respective first and second LED drive signals; at least first and second buffers coupled to the processor, wherein the processor is configured to selectively output (e.g., by way of at least one switch and control logic coupled between the processor and at least one of the first and second buffers) the photodiode current response to the first or second buffer based on the respective first and second LED drive signals; optionally at least one transimpedance amplifier coupled to the first and second buffers to convert the received photodiode response signal to respective voltage values (or signals), wherein the received photodiode response signal is a current signal; and radio communication circuitry (for example, comprising at least a modulator) coupled to the at least one buffers (and optionally also coupled to an antenna) configured to transmit the voltage data sets based on the buffered data.

Example 19. A method to be performed by a wireless transmitter connectable to a pulse oximeter unit, the method comprising: receiving a photodiode response signal from the pulse oximeter unit; generating a first data set comprising voltage values corresponding to a photodiode current sensed responsive to the first LED of the pulse oximeter unit based on the received photodiode response signal and a first LED drive signal (e.g., by way of buffering and optionally current to voltage conversion); generating a second data set comprising voltage values corresponding to a photodiode current sensed responsive to the second LED of the pulse oximeter unit based on the received photodiode response signal and a second LED drive signal (e.g., by way of buffering and optionally current to voltage conversion); generating a digitized and packetized signal comprising the first and second data sets; and wirelessly transmitting the signal (e.g., using radio communication protocols).

Example 20. The method of Example 19, wherein the method further comprises subtracting an ambient light signal from the respective first and second data sets.

Example 21. A system comprising: the receiver of any one of Examples 1 to 14; and the transmitter of Example 17 or Example 18.

Example 22. The system of Example 21, further comprising at least one of: a patient monitor, and a pulse oximeter unit.

Example 23. The system of Example 21 or Example 22, further comprising at least one of: two electrodes configured to provide an electro-cardiogram measurement, a temperature sensor, and a pulse oximeter.

Example 24. The system of any one of Examples 21 to 23, further comprising a transmitter configured to wirelessly transmit ECG and/or temperature sensor data.

Example 25. A receiver connectable to a patient monitor, the receiver comprising: a processor; and circuitry, coupled to the processor, the circuitry configured to: wirelessly receive electro-cardiogram, ECG, data sensed by an ECG unit and temperature sensor data sensed by a temperature sensor unit; generate at least a first signal based on the received ECG data, wherein the at least first signal is configured to emulate at least a first wired connection input signal received by the patient monitor from the ECG unit; generate an additional signal based on the temperature sensor data, wherein the additional signal is configured to emulate a wired connection input signal received by the patient monitor from the temperature sensor unit; and output the generated signals to the patient monitor.

Example 26. The receiver of Example 25, wherein the receiver is configured to wirelessly receive the ECG data and the temperature sensor data by wirelessly receiving at least digitized and packetized ECG and temperature sensor data in respective packets, wherein each packet has a corresponding header.

Example 27. The receiver of Example 25 or 26, wherein the temperature sensor data comprises a voltage signal based on a resistance controlled by the temperature sensor, and wherein the ECG data comprises a voltage signal representative of a difference in potential across two electrodes of the ECG unit.

Example 28. The receiver of Example 27, wherein the at least first generated signal is configured to generate a voltage signal across first and second inputs (e.g., input lines) of the patient monitor that are configured to receive the wired connection input from the ECG unit.

Example 29. The receiver of Example 27, wherein the additional signal is configured to emulate the current drawn from the patient monitor over a wired connection from the temperature sensor.

Example 30. The receiver of any one of Example 25 to 29, wherein the circuitry comprises: radio communication circuitry configured to wirelessly receive a digitized and packetized signal comprising the ECG and temperature sensor data; and signal generation circuitry configured to generate the at least first signal based on the received ECG data and the additional signal based on the received temperature sensor data.

Example 31. The receiver of Example 30, wherein the processor is configured to dynamically adapt the current drawn by the signal generation circuitry to at least one of: account for variation in the current at which the patient monitor drives the temperature sensor unit, and account for a difference between an intended current to be drawn from the patient monitor and an actual current drawn from the patient monitor.

Example 32. The receiver of Example 31, wherein signal generation circuitry is configured to provide at least one of: a feedback voltage signal to the processor to indicate the voltage at which the patient monitor drives the temperature sensor unit (or temperature sensor thereof), and a feedback current signal to the processor to indicate the actual (i.e., measured) current being drawn from the patient monitor.

Example 33. The receiver of any one of Examples 30 to 32, wherein the signal generation circuitry comprises a DAC coupled to a current generator configured to emulate the current drawn from the patient monitor over a wired connection from the temperature sensor unit based on the received temperature sensor data.

Example 34. The receiver of any one of Examples 30 to 33, the signal generation circuitry comprises current sensing circuitry configured to sense a current in a pathway of the signal generation circuitry and to provide the sensed current to the processor; the signal generation circuitry comprises voltage sensing circuitry configured to sense a voltage across the signal generation circuitry pathway and to provide the sensed voltage to the processor; and the processor is configured to: calculate, based on the sensed voltage and the temperature sensor data, a current to be drawn from the patient monitor, wherein the current to be drawn causes the signal generation circuitry pathway to have an impedance that emulates the resistance of a temperature sensor of the temperature sensor unit; and adjust, based on the sensed current, the current drawn from the patient monitor to draw the calculated current.

Example 35. The receiver of any one of Examples 30 to 34, wherein the receiver comprises: a voltage sensor coupled to an input of the processor; a current sensor coupled to an input of the processor; and a transistor (such as a MOSFET) coupled to an output of the processor (for example, by way of an digital to analogue converter).

Example 36. The receiver of any one of Examples 30 to 35, wherein the signal generation circuitry comprises: voltage scaling circuitry configured to scale a voltage signal based on the received ECG data such that the scaled voltage signal is centred around a given reference voltage; reference voltage signal generation circuitry configured to generate a constant reference voltage signal at a given reference voltage; wherein the voltage scaling circuitry and the reference voltage signal generation circuitry are configured, respectively, to provide the scaled voltage signal and the reference voltage signal to respective first and second input lines of the patient monitor configured to receive the wired connection input signals from the ECG unit.

Example 37. The receiver of Example 36, wherein the receiver comprises: a reference voltage supply; a voltage divider coupled to a first output of the reference voltage supply and buffer amplifier coupled to an output of the voltage divider; a digital to analogue converter coupled to an output of the processor and a second output of the reference voltage supply; and a summing amplifier coupled to an output of the DAC and a first output of the buffer amplifier, wherein a second output of the buffer amplifier is provided to a first input line of the patient monitor and wherein the output of the summing amplifier is provided to a second input line of the patient monitor.

Example 38. The receiver of Example 36 or 37, wherein the signal generation circuitry further comprises switching circuitry controlled by the processor to emulate a lead-off event at the patient monitor.

Example 39. The receiver of any one of Examples 36 to 38, wherein the processor is configured to control the switching circuitry to emulate a lead-off event in response to a detection that a lead-off event has occurred, for example that a lead-off event has been detected at a wireless transmitter connected to the ECG unit.

Example 40. The receiver of any one of Examples 36 to 39, wherein the receiver is further configured to wirelessly receive from the ECG unit at least one parameter value that describes an impedance signal waveform indicative of a respiratory measurement.

Example 41. The receiver of Example 40, wherein the receiver is configured to modulate the at least first generated signal to be output to the patient monitor (wherein the at least first generated signal is configured to emulate at least a first wired connection input signal received by the patient monitor from the ECG unit that indicates the electrical activity of the patient's heart) to emulate the impedance signal detected by the ECG unit (e.g., from the patient).

Example 42. The receiver of Example 40 or Example 41, wherein the signal generation circuitry is further configured to control an impedance of a signal generation circuitry pathway (e.g., between the processor and the first and second input lines of the patient monitor, or respective output lines, or output paths, of the receiver connectable to the input lines of the patient monitor) based on the received at least one parameter value.

Example 43. The receiver of Example 42, wherein the signal generation circuitry is configured to control an impedance of the signal generation circuitry pathway based on a voltage signal output by the processor, wherein processor is configured to generate the voltage signal to emulate at least one of a morphology and an amplitude of a waveform of the impedance signal detected by the ECG unit.

Example 44. The receiver of Example 42 or Example 43, wherein the signal generation circuitry comprises at least one resistive element configured to be controlled based on the received at least one parameter value to control the impedance in the signal generation circuitry pathway.

Example 45. The receiver of any one of Examples 42 to 44, wherein the signal generation circuitry comprises at least one transistor configured to control the impedance of the signal generation circuitry pathway based on a voltage signal provided by the processor to a gate thereof.

Example 46. The receiver of any one of Examples 42 to 45, wherein the signal generation circuitry comprises two back-to-back MOSFETS with a common gate, wherein the impedance of the signal generation circuitry pathway is controlled by a voltage signal output by the processor provided to the common gate.

Example 47. The receiver of any one of Examples 42 to 44, wherein the signal generation circuitry comprises a resistor network comprising a plurality of resistors connected in series and operable by way of a respective plurality of switches to control the impedance of the signal generation circuitry pathway based on the received at least one parameter value.

Example 48. The receiver of Example 47, wherein the resistor network comprises at least six resistors connected in series and operable by way of a respective plurality of switches.

Example 49. The receiver of Example 48, wherein the resistor network comprises eight resistors connected in series and operable by way of a respective plurality of switches.

Example 50. The receiver of any one of Examples 47 to 49, wherein the signal generation circuitry comprises at least one additional resistor coupled in series to the resistor network, for example, to modify the range of impedance values output by the plurality of resistors of the resistor network connected in series.

Example 51. The receiver of any one of Examples 47 to 50, wherein the signal generation circuitry further comprises at least one resistor connected in parallel to the resistor network, for example, to rescale the impedance output from the resistor network.

Example 52. A method to be performed by a receiver connectable to a patient monitor, the method comprising: wirelessly receiving electro-cardiogram, ECG, data sensed by an ECG unit and temperature sensor data sensed by a temperature sensor unit; generating at least a first signal based on the received ECG data, wherein the at least first signal is configured to emulate at least a first wired connection input signal received by the patient monitor from the ECG unit; generating an additional signal based on the temperature sensor data, wherein the additional signal is configured to emulate a wired connection input signal received by the patient monitor from the temperature sensor unit; and outputting the generated signals to the patient monitor.

Example 53. The method of Example 52, wherein wirelessly receiving the ECG data and the temperature sensor data comprises wirelessly receiving at least digitized and packetized ECG and temperature sensor data in respective packets, wherein each packet has a corresponding header.

Example 54. The method of Example 52 or Example 53, wherein generating the at least a first signal based on the received ECG data comprises: scaling a voltage signal based on the received ECG data such that the scaled voltage signal is centred around a given reference voltage; generating a constant reference voltage signal at the given reference voltage; and providing the scaled voltage signal and the reference voltage signal to respective first and second connectors of the patient monitor configured to receive the wired connection input signals from the ECG unit.

Example 55. The method of any one of Examples 52 to 54, wherein the method further comprises controlling by the processor of the receiver switching circuitry to emulate a lead-off event at the patient monitor. For example, controlling by the processor the switching circuitry to emulate a lead-off event in response to detecting a lead-off characteristic in the ECG data.

Example 56. The method of any one of Examples 52 to 55, wherein generating the additional signal comprises: sensing a current in the signal generation circuit pathway between the processor of the receiver and a connector of the patient monitor configured to receive the wired connection input signal from the temperature sensor unit, and providing the sensed current to the processor; sensing a voltage across the signal generation circuit pathway between the processor and the connector of the patient monitor and providing the sensed voltage to the processor; calculating, by the processor, based on the sensed voltage and the temperature sensor data, a current to be drawn from the patient monitor, wherein the current to be drawn causes the signal generation circuit pathway to have an impedance that emulates the resistance of a temperature sensor of the temperature sensor unit; and adjusting, based on the sensed current, the current drawn from the connector of the patient monitor to draw (e.g., to substantially match) the calculated current.

Example 57. The method of any one of Examples 52 to 56, wherein generating the additional signal comprises providing a feedback signal (e.g., a feedback current signal) to the processor.

Example 58. The method of any one of Examples 52 to 57, wherein the method further comprises receiving, from an ECG unit, at least one parameter value that describes an impedance signal waveform indicative of a respiratory measurement; and controlling an impedance of a signal generation circuitry pathway of the receiver based on the received at least one parameter value.

Example 59. The method of claim 58, wherein controlling the impedance of the signal generation circuitry pathway of the receiver based on the received at least one parameter value comprises controlling a resistor network based on the received at least one parameter value (or a voltage signal generated therefrom).

Example 60. A transmitter connectable to an ECG unit and a temperature sensor unit, the transmitter comprising a processor; and circuitry coupled to the processor, the circuitry configured to: receive ECG data from the ECG unit; receive temperature sensor data from the temperature sensor unit; generate a digitized and packetized signal comprising the ECG and temperature sensor data; and wirelessly transmit the signal.

Example 61. The transmitter of Example 60, wherein the signal comprises ECG data and temperature sensor data packetized in respective packets, and wherein each packet has a corresponding header.

Example 62. The transmitter of Example 60 or 61, wherein the circuitry is further configured to transmit at least one parameter value that describes an impedance signal waveform indicative of a respiratory measurement, and is optionally configured to determine the at least one parameter value.

Example 63. A method to be performed by a transmitter connectable to an ECG unit and a temperature sensor unit, the method comprising: receiving ECG data from the ECG unit; receiving temperature sensor data from the temperature sensor unit; generating a digitized and packetized signal comprising the ECG and temperature sensor data; and wirelessly transmitting the signal.

Example 64. The method of Example 63, wherein the method further comprises: transmitting at least one parameter value that describes an impedance signal waveform indicative of a respiratory measurement, and optionally further comprises determining the at least one parameter value.

Example 65. A system comprising: the receiver of any one Examples 25 to 51; and the transmitter of any one of Examples 60 to 63.

Example 66. The system of Example 65 further comprising at least one of: at least two electrodes configured to provide an electro-cardiogram measurement, a temperature sensor, and a pulse oximeter.

Example 67. The use of any one of the preceding Examples in monitoring at least one vital sign of an infant, for example an infant in neonatal care.

It should be understood that the examples of the devices described above may be combined in any combination where feasible.

It should also be understood that the examples of the methods described above may be combined in any combination where feasible.

1. A receiver connectable to a patient monitor, the receiver comprising:
a processor; and
circuitry, coupled to the processor, the circuitry configured to:
wirelessly receive electro-cardiogram, ECG, data sensed by an ECG unit and temperature sensor data sensed by a temperature sensor unit;
generate at least a first signal based on the received ECG data, wherein the at least first signal is configured to emulate at least a first wired connection input signal received by the patient monitor from the ECG unit;
generate an additional signal based on the temperature sensor data, wherein the additional signal is configured to emulate a wired connection input signal received by the patient monitor from the temperature sensor unit; and
output the generated signals to the patient monitor.

2. The receiver of claim 1, wherein the receiver is configured to wirelessly receive the ECG data and the temperature sensor data by wirelessly receiving at least digitized and packetized ECG and temperature sensor data in respective packets, wherein each packet has a corresponding header.

3-13. (canceled)

14. A method to be performed by a receiver connectable to a patient monitor, the method comprising:
wirelessly receiving electro-cardiogram, ECG, data sensed by an ECG unit and temperature sensor data sensed by a temperature sensor unit;
generating at least a first signal based on the received ECG data, wherein the at least first signal is configured to emulate at least a first wired connection input signal received by the patient monitor from the ECG unit;
generating an additional signal based on the temperature sensor data, wherein the additional signal is configured to emulate a wired connection input signal received by the patient monitor from the temperature sensor unit; and
outputting the generated signals to the patient monitor.

15. (canceled)

16. A transmitter connectable to an ECG unit and a temperature sensor unit, the transmitter comprising:
a processor; and
circuitry coupled to the processor, the circuitry configured to:
receive ECG data from the ECG unit;
receive temperature sensor data from the temperature sensor unit;
generate a digitized and packetized signal comprising the ECG and temperature sensor data; and
wirelessly transmit the signal.

17. (canceled)

18. A system comprising:
a receiver connectable to a patient monitor; and
a transmitter connectable to an ECG unit and a temperature sensor unit,
wherein the receiver comprises:
a processor; and
circuitry, coupled to the processor, the circuitry configured to:
wirelessly receive electro-cardiogram, ECG, data sensed by an ECG unit and temperature sensor data sensed by a temperature sensor unit;
generate at least a first signal based on the received ECG data, wherein the at least first signal is configured to emulate at least a first wired connection input signal received by the patient monitor from the ECG unit;
generate an additional signal based on the temperature sensor data, wherein the additional signal is configured to emulate a wired connection input signal received by the patient monitor from the temperature sensor unit; and
output the generated signals to the patient monitor; and
wherein the transmitter comprises:
a processor; and
circuitry coupled to the processor, the circuitry configured to:
receive ECG data from the ECG unit;
receive temperature sensor data from the temperature sensor unit;
generate a digitized and packetized signal comprising the ECG and temperature sensor data; and
wirelessly transmit the signal.

19. (canceled)

20. A receiver connectable to a patient monitor, the receiver comprising:
a processor; and
circuitry coupled to the processor, the circuitry configured to:
wirelessly receive data based on an output from a pulse oximeter unit, wherein the data comprises:
a first data set comprising voltage values corresponding to a photodiode current sensed responsive to a first LED of the pulse oximeter unit, and
a second data set comprising voltage values corresponding to a photodiode current sensed responsive to a second LED of the pulse oximeter unit;
generate a signal based on the received data, wherein the signal is configured to emulate a wired connection input signal to the patient monitor; and
output the generated signal to the patient monitor.

21. The receiver of claim 20, wherein the receiver is further configured to:
generate a load to emulate a load on the patient monitor by the first and second LEDs of the pulse oximeter unit; and
apply the load to the patient monitor.

22. The receiver of claim 20, wherein the circuitry comprises:
radio communication circuitry configured to wirelessly receive a digitized and packetized signal comprising the data from the pulse oximeter unit; and
signal generation circuitry configured to generate the signal to be output to the patient monitor, wherein said signal generation circuitry comprises switching circuitry configured to be controlled to selectively provide the signal to the patient monitor based on a respective one of the first and second data sets of voltage values.

23. The receiver of claim 22, wherein the signal generation circuitry comprises optocoupler circuitry comprising a photodiode configured to provide a current to the patient monitor.

24. The receiver of claim 22, wherein the signal generation circuitry comprises:
digital-to-analogue conversion, DAC, circuitry configured to convert the respective received first and second voltage data sets to respective first and second analogue voltage signals;
LED signal detection circuitry configured to detect respective first and second LED drive signals from the patient monitor, wherein the first and second LED drive signals correspond to the first and second LEDs of the pulse oximeter unit respectively;
control logic; and
optocoupler circuitry;
wherein the LED signal detection circuitry is configured to provide the first and second LED drive signals to the control logic,
wherein the control logic is configured to select which of the first or second analogue voltage signals to provide to the optocoupler circuitry at a given timestep based on the detected first and second LED drive signals, and
wherein the optocoupler circuitry is driven based on the selected analogue voltage signal and is configured to provide an emulated wired connection photodiode response to the patient monitor.

25. The receiver of claim 24, wherein the first and second LED drive signals are respective first and second LED drive phase signals.

26. The receiver of claim 24, wherein the LED signal detection circuitry is further configured to provide a current feedback signal to the processor.

27. The receiver of claim 24, wherein the control logic is further configured to select an analogue voltage signal corresponding to the photodiode response to an ambient light signal to provide at a given timestep as output to the optocoupler circuitry responsive to an indication that neither the first nor second LED of the pulse oximeter unit are being driven by the patient monitor at that timestep.

28. The receiver of claim 22, wherein the signal generation circuitry further comprises respective first and second LEDs to emulate the load on the patient monitor by the first and second LEDs of the pulse oximeter unit.

29. The receiver of claim 28, wherein the first and second LEDs are connectable to first and second output lines of the patient monitor, and a photodiode of the optocoupler circuitry of the signal generation circuitry is connectable to a first input line of the patient monitor.

30. A method to be performed by a receiver connectable to a patient monitor, the method comprising:
wirelessly receiving data based on an output from a pulse oximeter unit, wherein the data comprises:
a first data set comprising voltage values corresponding to a photodiode current sensed responsive to a first LED of the pulse oximeter unit, and
a second data set comprising voltage values corresponding to a photodiode current sensed responsive to a second LED of the pulse oximeter unit;
generating a signal based on the received data wherein the signal is configured to emulate a wired connection input signal to the patient monitor; and
outputting the generated signal to the patient monitor.

31. The method of claim 30, further comprising:
generating, by the receiver, a load to emulate a load on the patient monitor by the first and second LEDs of the pulse oximeter; and
applying the load to the patient monitor.

32. A transmitter connectable to a pulse oximeter unit, the transmitter comprising:
a processor; and
circuitry, coupled to the processor, the circuitry configured to:
receive a photodiode response signal from the pulse oximeter unit;
generate a first data set comprising voltage values corresponding to a photodiode current sensed responsive to a first LED of the pulse oximeter unit based on the received photodiode response signal and a first LED drive signal;
generate a second data set comprising voltage values corresponding to a photodiode current sensed responsive to a second LED of the pulse oximeter unit based on the received photodiode response signal and a second LED drive signal;
generate a digitized and packetized signal comprising the first and second data sets; and
wirelessly transmit the signal.

33. A system comprising:
a receiver connectable to a patient monitor; and
a transmitter connectable to a pulse oximeter unit,
wherein the receiver comprises:
a processor; and
circuitry coupled to the processor, the circuitry configured to:
wirelessly receive data based on an output from a pulse oximeter unit, wherein the data comprises:
a first data set comprising voltage values corresponding to a photodiode current sensed responsive to a first LED of the pulse oximeter unit, and
a second data set comprising voltage values corresponding to a photodiode current sensed responsive to a second LED of the pulse oximeter unit;
generate a signal based on the received data, wherein the signal is configured to emulate a wired connection input signal to the patient monitor; and
output the generated signal to the patient monitor, and
wherein the transmitter comprises:
a processor; and
circuitry, coupled to the processor, the circuitry configured to:
receive a photodiode response signal from the pulse oximeter unit;
generate a first data set comprising voltage values corresponding to a photodiode current sensed responsive to a first LED of the pulse oximeter unit based on the received photodiode response signal and a first LED drive signal;
generate a second data set comprising voltage values corresponding to a photodiode current sensed responsive to a second LED of the pulse oximeter unit based on the received photodiode response signal and a second LED drive signal;
generate a digitized and packetized signal comprising the first and second data sets; and
wirelessly transmit the signal.

34. The system of claim 33, further comprising at least one of: a pulse oximeter, at least two electrodes configured to provide an electro-cardiogram measurement, and a temperature sensor.
